(19) United States
(12) Patent Application Publication
(10) Pub. No.: US 2019/0119377 A1
(43) Pub. Date: Apr. 25, 2019

(54) RECOMBINANT IGG FC MULTIMERS

(71) Applicant: CSL BEHRING RECOMBINANT FACILITY AG, Bern (CH)

(72) Inventors: Rolf SPIRIG, Bern (CH); Fabian KAESERMANN, Burgdorf (CH); Adrian ZUERCHER, Bern (CH); Con PANOUSIS, Bundoora (AU); Adriana BAZ MORELLI, Carlton (AU); Chao-Guang CHEN, Parkville (AU)

(21) Appl. No.: 16/072,678

(22) PCT Filed: Jan. 27, 2017
(86) PCT No.: PCT/EP2017/051757

§ 371 Date: Jul. 25, 2018

(30) Foreign Application Priority Data
Jan. 27, 2016 (EP) 16152867.4
Mar. 24, 2016 (EP) 16162166.9
Oct. 21, 2016 (EP) 16195116.5

(51) Int. Cl. C07K 16/28; A61P 37/00; A61K 9/00

(52) U.S. Cl. CPC C07K 16/2803; A61K 9/0019; A61P 37/00

(57) ABSTRACT

This disclosure provides recombinant IgG Fc multimers and methods of treating autoimmune and inflammatory diseases by administering such multimers.

This application is the United States national stage entry under 35 U.S.C. § 371 of International Application No. PCT/EP2017/051757, filed on Jan. 27, 2017, which claims priority to European Patent Application Nos. 16195116.5, filed on Oct. 21, 2016, 16162166.9, filed on Mar. 24, 2016 and 16152867.4, filed on Jan. 27, 2016. The contents of these applications are each incorporated herein by reference in their entirety.

### FIELD

This disclosure provides recombinant IgG Fc multimers and methods of treating autoimmune and inflammatory diseases by administering such multimers.

### BACKGROUND

Plasma-derived immunoglobulin G (IgG) is used in the clinics to treat primary and secondary immunodeficiency. In this case, IgG is administered either intravenously (IVIG) or subcutaneously (SCIG). Both are prepared from large plasma pools of more than 10,000 donors, ensuring a diverse antibody repertoire.

However, the administration of high doses of IVIG (1-2 g/kg/dose) has been increasingly used for the treatment of patients with chronic or acute autoimmune and inflammatory diseases such as immune cytopenia (ITP), Guillain-Barré syndrome, Kawasaki disease, chronic inflammatory demyelinating polyneuropathy (CIDP), myasthenia gravis (MG), and several other rare diseases. Additionally, off-label uses of IVIG for several other indications are currently under exploration such as, for example, for the treatment of rheumatoid arthritis (RA).

Numerous mechanisms of action have been proposed for the anti-inflammatory effect of high-dose IVIG. These include blockage of Fcγ receptors (FcγRs), saturation of neonatal FcR (FcRn) to enhance autoantibody clearance, up-regulation of inhibitory FcγRIIB (CD32B), scavenging of complement protein fragments and inhibition of complement fragment deposition, anti-idiotypic antibodies (Abs) in IVIG, binding or neutralization of immune mediators (e.g. cytokines), or modulation of immune cells (e.g. induction of regulatory T cells, B cells or tolerogenic dendritic cells). Interestingly, several of the above mentioned properties could be recapitulated with only the Fc portion of IgG. Fc has been shown to be therapeutic in several animal models of disease as e.g. ITP as well as inflammatory arthritis. Furthermore, Fc has been demonstrated to be therapeutically active in children with acute ITP suggesting critical involvement of FcγRs.

Several recent studies investigated the efficacy of recombinant Fc protein based therapeutics. In particular, multimerization of Fc resulting in polyvalent molecules with high avidity to FcγRs has been investigated.

Prospective IVIG replacement proteins comprising multiple serial linked Fc domains are described in WO 2008/151088. The serial Fc domains are described as further linked to the IgM tailpiece and J chain is incorporated in order to produce Fc pentameric structures. However, no working examples are provided regarding the preparation or efficacy of the envisaged multimeric proteins.

Sorensen et al. (1996) J Immunol 156: 2858-2856 describes several IgG multimeric constructs comprising CH1, CH2, and CH3 IgG domains fused to an IgM tailpiece. Other constructs had an IgM domain substituted for one or more of the IgG heavy chain domains. The study focused on determining the influence of J chain on the multimerization of various tailpiece constructs and found that constructs comprising mostly IgG heavy chain domains polymerized without incorporation of J chain. The study also showed that a L309C residue change in the IgG Fc domain resulted in higher order multimers, predominantly hexamers.

WO 2015/132364 and WO 2015/132365 disclose several Fc multimeric constructs comprising a five amino acid hinge region, an Fc region derived from IgG1, IgG4, or a hybrid of IgG1 and IgG4 CH2 and CH3 domains, and an IgM or IgA tailpiece. The disclosures are directed to improving safety and efficacy of IgG Fc multimers through the introduction of amino acid changes in the Fc regions of the fusion peptides. Preferred mutations improved binding to FcRn, increased multimerization of the Fc fusion monomers, and decreased Fc multimer binding to complement pathway component C1q. Fc multimers were also engineered with mutations to reduce stimulation of cytokine release and platelet activation.

WO 2014/060712 discloses an Fc multimeric construct comprising an IgG1 Fc region, a four amino acid linker, and an IgM tailpiece, which multimerizes to predominantly hexameric structure. Mutations at Fc residues 309 and 310 (L309C and H310L) were introduced to mimic the sequence of IgM. As expected, due to the lack of a Fab region, the mutant Fc hexamer bound poorly to C1q and failed to activate the complement system, as shown by a lack of detection of the C5b-9 terminal complex. The disclosed Fc multimer was effective in restoring platelet counts in a mouse model of ITP.

The inventors have found that the optimized hexameric Fc-μTP constructs of the invention have several benefits over those described previously:
Surprisingly, Fc-μTP and Fc-μTP-L309C bound C1q, but did not induce cleavage of the complement protein C2 and therefore no C3 convertase was formed (C4b2a).
Fc-μTP and Fc-μTP-L309C selectively inhibit activation of the complete classical pathway.
No interference with the alternative pathway was observed and therefore this complement pathway is still intact for defense against infectious diseases. No systemic blockage of the complement system occurred.
Fc-μTP and Fc-μTP-L309C might be especially beneficial in diseases which are auto-antibody mediated and/or in which antibody-mediated classical pathway activation of the complement system occurs.

### SUMMARY

The present disclosure provides an Fc multimeric protein, comprising two to six IgG Fc fusion monomers. Each of the IgG Fc fusion monomers comprises two Fc fusion polypeptide chains and each Fc fusion polypeptide chain comprises an IgG Fc polypeptide and an IgM tailpiece. In a preferred embodiment, the Fc multimer is an Fc hexamer, comprising six IgG Fc fusion monomers.

In a preferred embodiment, the Fc fusion polypeptide chain further comprises an IgG hinge region and the Fc fusion polypeptide chain does not comprise a Fab polypeptide.

For example, in one embodiment of the invention, the Fc fusion polypeptide chain of the invention comprises an IgG1 hinge region, an IgG1 Fc domain, and an IgM tailpiece, and does not comprise a Fab polypeptide. In a preferred embodiment, the Fc fusion polypeptide chain is SEQ ID NO:1 and has up to 5 conservative amino acid changes. In one embodiment the Fc fusion polypeptide chain is SEQ ID NO:7.

In a preferred embodiment, the Fc fusion polypeptide chain comprises an IgG1 hinge region, an IgG1 Fc domain, and an IgM tailpiece, wherein the IgG1 Fc domain has a cysteine instead of a leucine at position 309 (according to the EU numbering), and wherein the Fc fusion polypeptide does not comprise a Fab polypeptide and the Fc fusion polypeptide chain is SEQ ID NO:2. In one embodiment, the Fc fusion polypeptide chain is SEQ ID NO:2 with up to 5 conservative amino acid changes. In one embodiment the Fc fusion polypeptide chain is SEQ ID NO:8.

The Fc fusion polypeptide chain as synthesized within a host cell will additionally comprise a signal peptide, e.g. a signal peptide with a sequence shown in SEQ ID NO:4. The signal peptide is however cleaved off during secretion, and therefore typically no longer present in the resulting mature Fc hexamer.

A further embodiment of the invention is a polynucleotide encoding the Fc fusion polypeptide chain, preferably the polynucleotide also encodes a signal peptide linked to the Fc fusion polypeptide chain.

In a preferred embodiment, the Fc hexamer binds complement component C1q. In one embodiment, the Fc hexamer binding to C1q does not induce activation of the complete classical complement pathway.

In a preferred embodiment, the Fc hexamer binding to C1q does not induce cleavage of the majority of complement pathway component C2.

In a preferred embodiment, the Fc hexamer does not induce cleavage of C2.

In a preferred embodiment, the Fc hexamer binding to C1q does not result in formation of the complement pathway component C3 convertase.

In a preferred embodiment, the Fc hexamer does not induce formation of the complement pathway component soluble C5b-9. In one embodiment, 1 mg/ml of the Fc hexamer incubated with whole blood induces less than 20% of soluble C5b-9 generation as compared to soluble C5b-9 generation induced by heat-aggregated IgG incubated with whole blood. In one embodiment, the Fc hexamer incubated with whole blood induces less than 10% of soluble C5b-9 generation as compared to soluble C5b-9 generation induced by heat-aggregated IgG incubated with whole blood.

In a preferred embodiment, the Fc hexamer inhibits C5b-9 generation. In one embodiment, the Fc hexamer inhibits soluble C5b-9 generation by heat-aggregated IgG incubated with whole blood.

In a preferred embodiment, administration of the Fc hexamer in a mouse arthritis model induces a reduction in the clinical score, number of joint infiltrating cells, or histological score compared to untreated arthritic mice. In one embodiment, administration of 200 mg/kg of the Fc hexamer at day 6 in a mouse anti-collagen antibody-induced arthritis model induces a reduction in the clinical score at any of the days 7 to 14; a reduction in the mean clinical score calculated from days 7 to 14; a reduction in the number of CD45+ cells recovered from knee joints at day 8; or a reduction of histological score of ankle joints at day 8 or day 14, compared to untreated arthritic mice. In a preferred embodiment, administration of 200 mg/kg of the Fc hexamer at day 6 in a mouse anti-collagen antibody-induced arthritis model induces a greater than 50% reduction in the clinical score at any of the days 7 to 14; a greater than 50% reduction in the mean clinical score calculated from days 7 to 14; a greater than 50% reduction in the number of CD45+ cells recovered from knee joints at day 8; or a greater than 25% reduction of histological score of ankle joints at day 8 and/or a greater than 50% reduction of histological score of ankle joints at day 14, compared to untreated arthritic mice.

In a preferred embodiment, the Fc hexamer inhibits lysis of opsonized red blood cells in a hemolysis assay for the classical complement pathway as compared to recombinant monomeric Fc fragments. In one embodiment, the Fc hexamer inhibits lysis of opsonized sheep red blood cells in a hemolysis assay for the classical complement pathway as compared to recombinant Fc monomer of SEQ ID NO:3. In one embodiment, a concentration of 0.5 mg/ml of the Fc hexamer inhibits lysis of opsonized sheep red blood cells in a hemolysis assay for the classical complement pathway by over 70% as compared to a recombinant Fc monomer comprising two polypeptides of SEQ ID NO:3.

In a preferred embodiment, the Fc hexamer induces a reduction of Fcγ receptor II expression or Fcγ receptor III expression on neutrophils or monocytes. In one embodiment, administration of 200 mg/kg of the Fc hexamer at day 6 in a mouse anti-collagen antibody-induced arthritis model induces a greater than 50% reduction in Fcγ receptor II or Fcγ receptor III levels on neutrophils or monocytes at day 8 compared to untreated arthritic mice.

In a preferred embodiment, the Fc hexamer inhibits upregulation of C5a receptor (CD88) on monocytes.

In a preferred embodiment, the Fc hexamer induces a reduction of Fcγ receptor I (CD64) levels on monocytes. In one embodiment, administration of 200 mg/kg of the Fc hexamer at day 6 in a mouse anti-collagen antibody-induced arthritis model induces a reduction in Fcγ receptor I (CD64) on monocytes at day 8 compared to untreated arthritic mice.

In a preferred embodiment, the Fc hexamer functionally blocks neonatal Fc receptors (FcRn) in vivo. In one embodiment, administration of 200 mg/kg of the Fc hexamer significantly increases the clearance of a tracer antibody in a transgenic mouse expressing human FcRn.

In a preferred embodiment administration of 200 mg/kg of the Fc hexamer decreases the half-life of a tracer antibody by at least 20%, more preferably by at least 30%, 40%, 50%, even more preferably by at least 60%.

In a preferred embodiment, the Fc hexamer inhibits the phagocytosis of human IgG-coated beads by THP-1 cells, indicating inhibition of receptor-mediated phagocytosis, more efficiently than equivalent concentrations of IVIG. In a preferred embodiment a 10-fold lower concentration of the Fc hexamer as compared to the IVIG concentration is able to achieve at least the same level of inhibition as is achieved with the IVIG concentration used, preferably a 30-fold lower concentration of the Fc hexamer, even more preferably a 100-fold lower concentration, even more preferably a 300-fold lower concentration, most preferably a 1000-fold lower concentration of the Fc hexamer as compared to the IVIG concentration is able to achieve at least the same level of inhibition as the IVIG concentration used.

In a preferred embodiment, the Fc hexamer does not activate human neutrophils in vitro, as shown by lack of calcium mobilization in response to concentrations up to 3 mg/ml. In a preferred embodiment calcium mobilization by Fc hexamer in human neutrophils is less than 50%, more preferably less than 40%, less than 30%, even more preferably less than 20%, most preferably less than 10% of the calcium mobilization observed with heat-aggregated IgG.

In a preferred embodiment, the Fc hexamer inhibits activation measured calcium mobilization of neutrophils by heat aggregated IgG, simulating immune complexes, more efficiently than IVIG. using a 2-fold lower concentration of Fc hexamer than IVIG, preferably using a 4-fold lower concentration, even more preferably using an 8-fold lower concentration of Fc hexamer than of IVIG.

In a preferred embodiment an equivalent level of inhibition of respiratory burst induced by IgG coated RBC by Fc hexamer is achieved using a 2-fold lower concentration of Fc hexamer than monomeric Fc, preferably using a 4-fold lower concentration, even more preferably using an 8-fold lower concentration of Fc hexamer than of monomeric Fc.

In preferred embodiment calcium mobilization in human monocytes in presence of high normal human serum concentrations (i.e. >20%) by Fc hexamer is less than 40%, preferably less than 30%, more preferably less than 20%, even more preferably less than 10% of the calcium mobilization observed with a concentration of heat aggregated IgG that achieves maximal stimulation.

The present disclosure also provides a method for treating an autoimmune or inflammatory disease in a subject by administering a therapeutically effective amount of a pharmaceutical composition of the Fc hexamer to a subject in need thereof.

In a preferred embodiment, the Fc hexamer is administered intravenously or non-intravenously. In one embodiment, the Fc hexamer is administered subcutaneously. In one embodiment, the Fc hexamer is applied orally, or intrathecally, or intrapulmonarily by nebulization.

In a preferred embodiment, the autoimmune or inflammatory disease is chosen from immune cytopenia, Guillain-Barré syndrome, Kawasaki disease, chronic inflammatory demyelinating polyneuropathy, myasthenia gravis, inflammatory neuropathy, neuromyelitis optica, other autoimmune channelopathies, autoimmune epilepsy, dermatomyositis or polymyositis, pemphigus or pemphigoid, scleroderma, systemic lupus erythematosus and rheumatoid arthritis. In one embodiment, the autoimmune diseases are auto-antibody mediated. In one embodiment, the inflammatory disease is linked to transplantation. In one embodiment the inflammatory disease is due to reperfusion injury or the inflammation is due to spinal cord injury.

In a preferred embodiment the Fc hexamer is administered in an amount ranging from about 10 mg/kg to about 200 mg/kg. In one embodiment, the Fc hexamer is administered in an amount ranging from about 25 mg/kg to about 500 mg/kg. All doses are per kg of bodyweight of the subject to which the Fc hexamer is administered.

It is to be understood that the foregoing general description and the following detailed description are exemplary and explanatory only, and are intended to provide further, non-limiting explanation of the disclosure.

### BRIEF DESCRIPTION OF DRAWING(S)

FIG. 1A shows a schematic diagram of Fc-μTP and Fc-μTP-L309C hexamer structures.

FIG. 1B shows SDS PAGE of Fc-μTP (left) and Fc-μTP-L309C (right) Fc proteins. Molecular weight markers in kDa are shown.

FIG. 10 shows the size exclusion chromatography (SEC) of Fc-μTP (left) and Fc-μTP-L309C (right). Chromatograms show the normalized U.V. absorbance signals at 280 nm (A280) and the thick bold lines show the molecular weight (in kDa) of material eluted at the time indicated, determined by multi-angle light scattering (MALS).

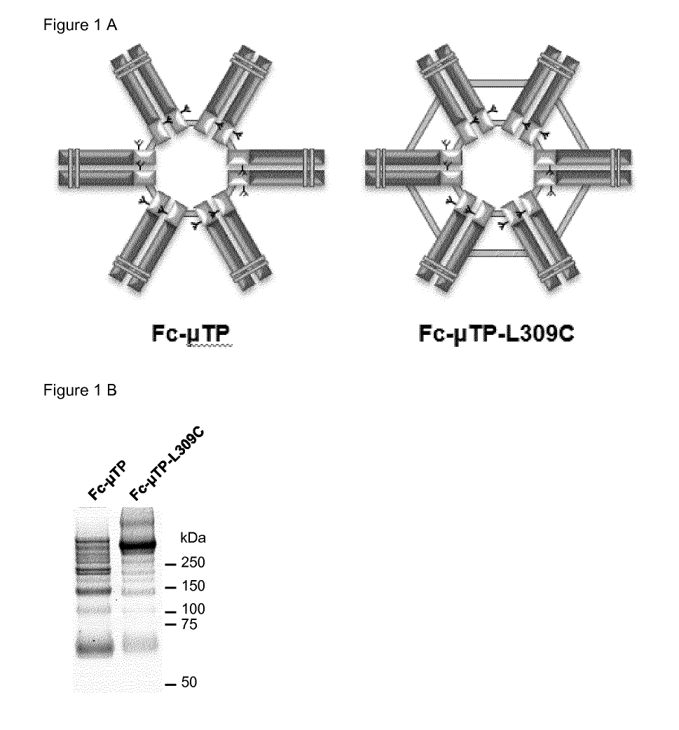

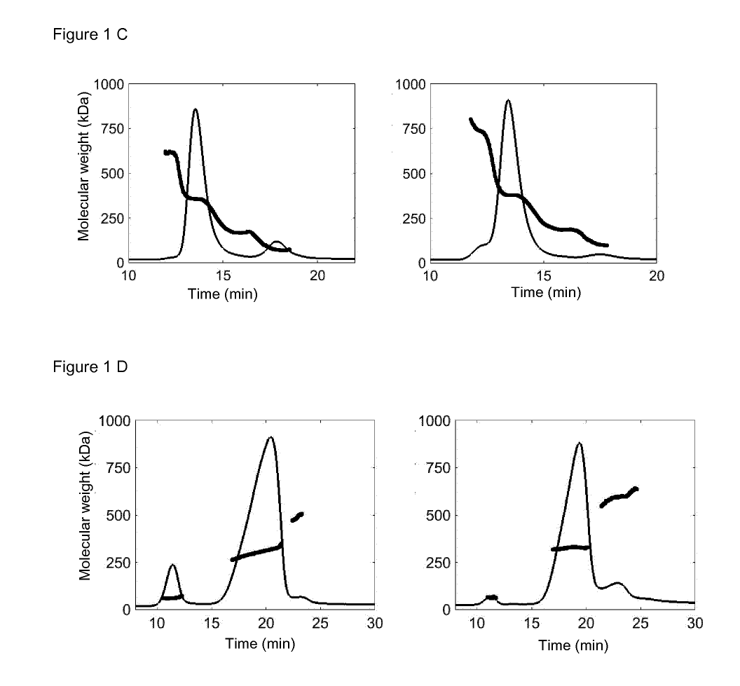

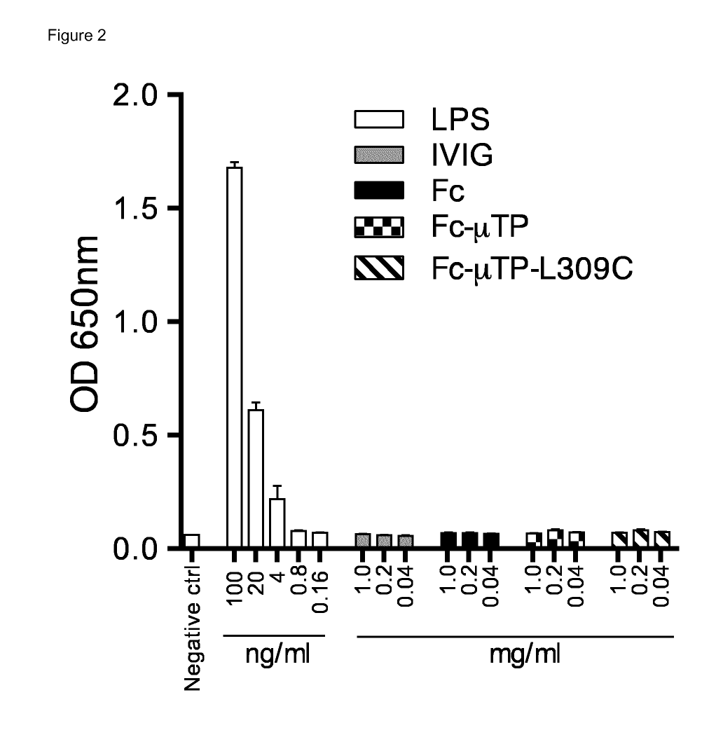

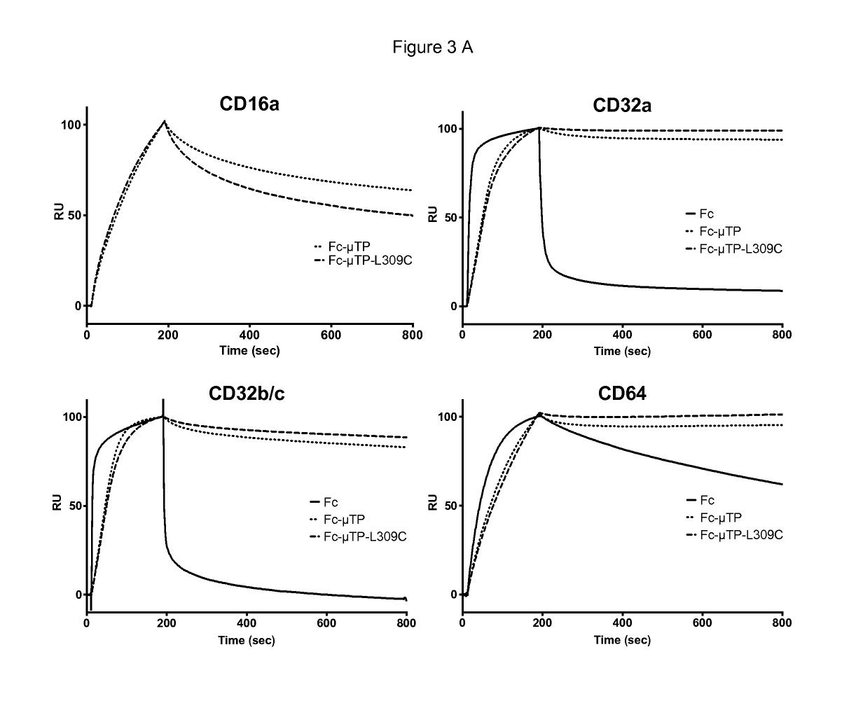

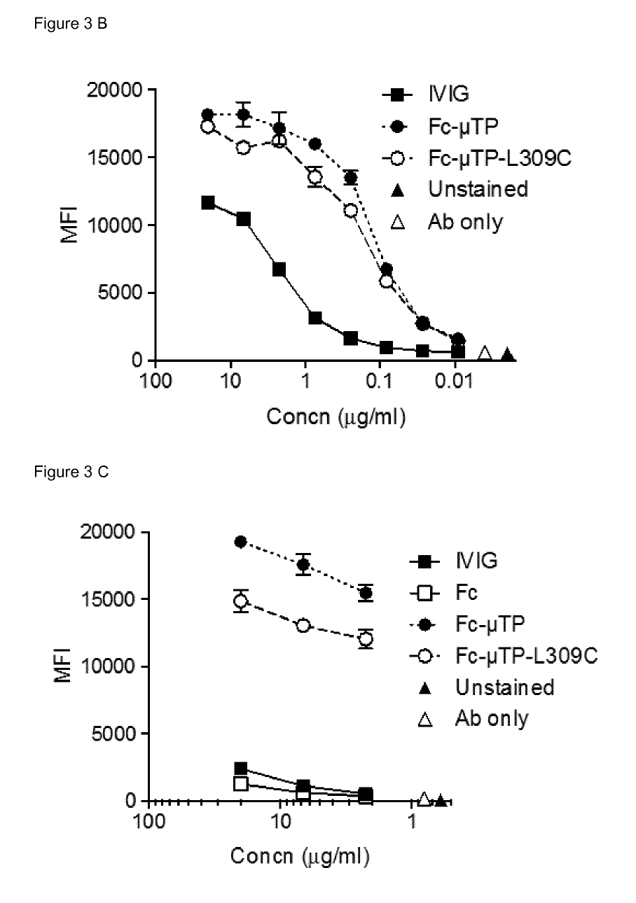

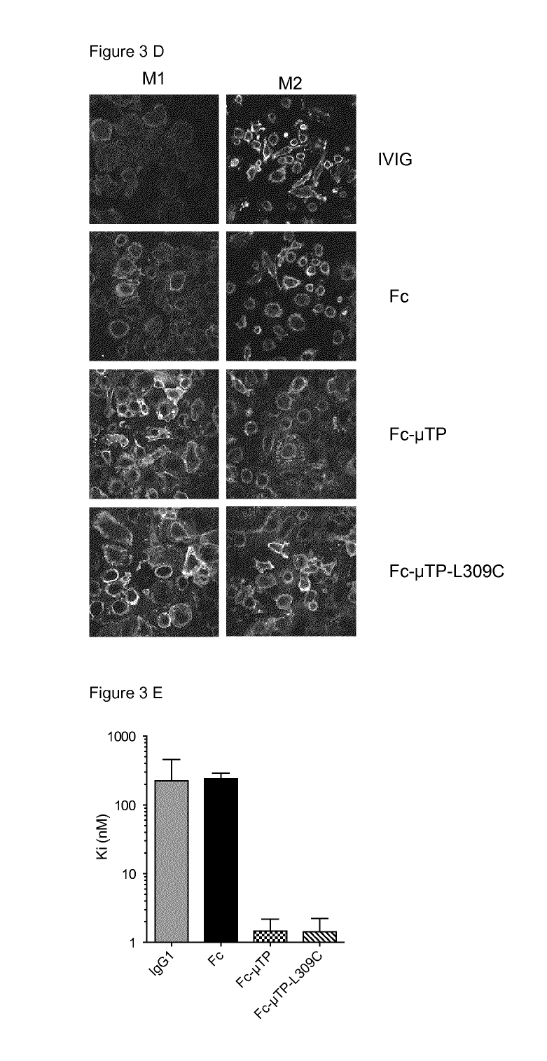

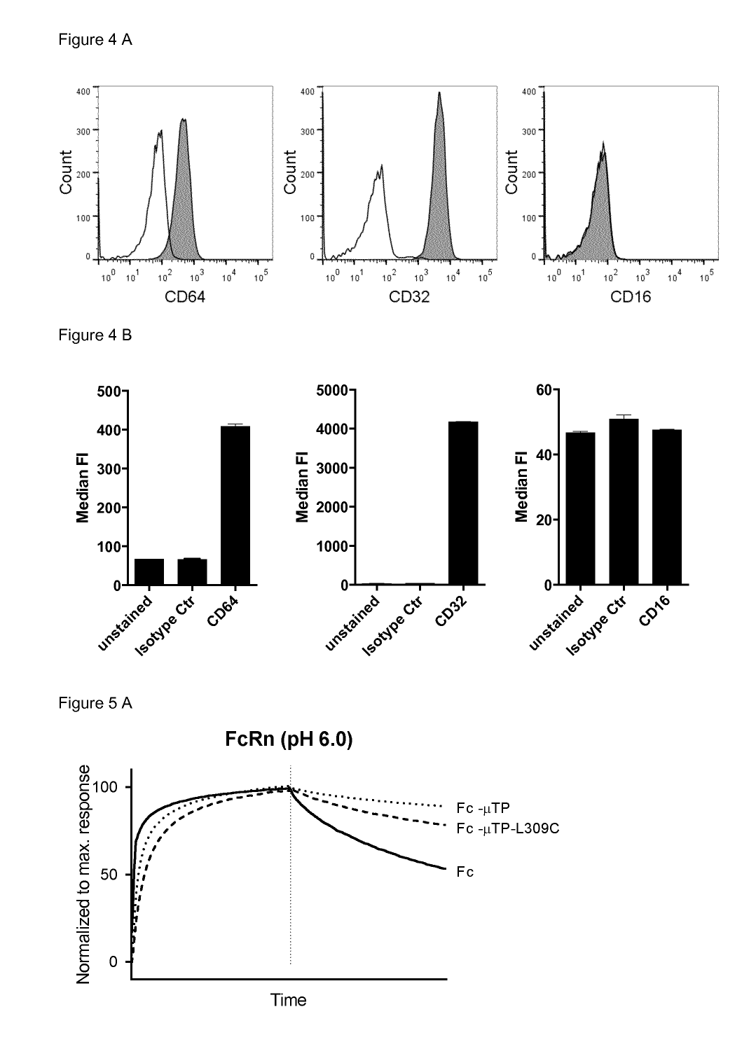

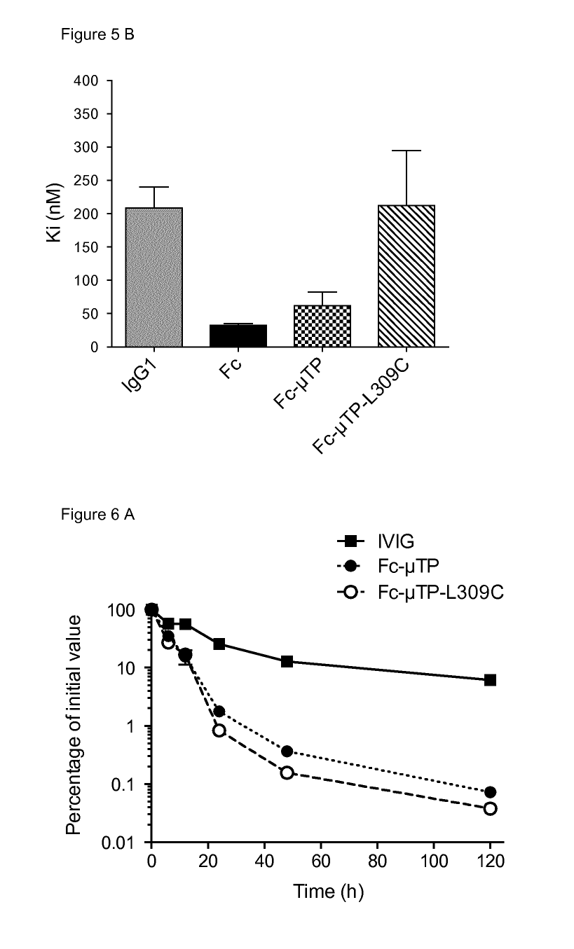

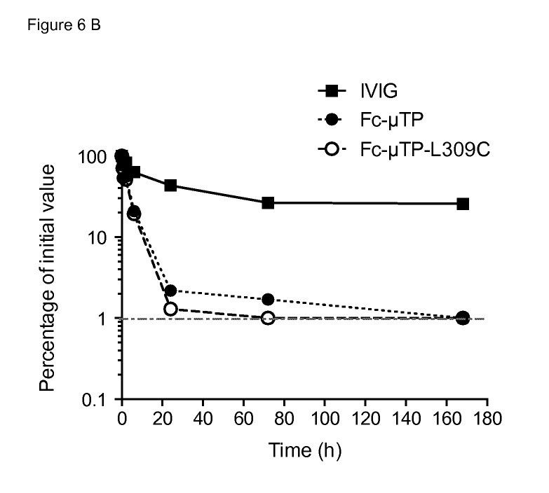

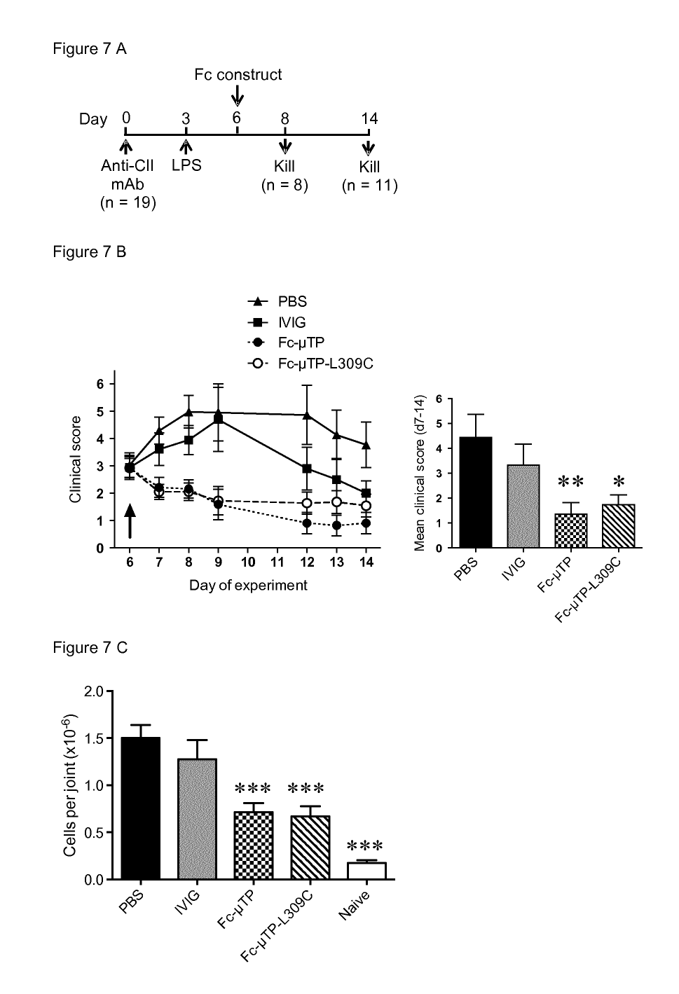

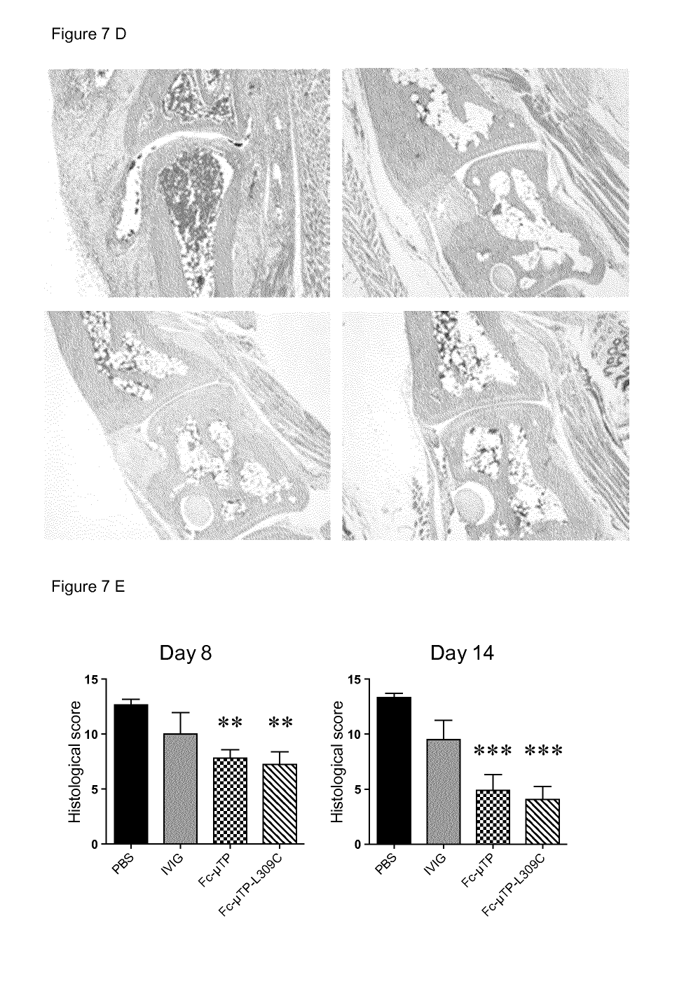

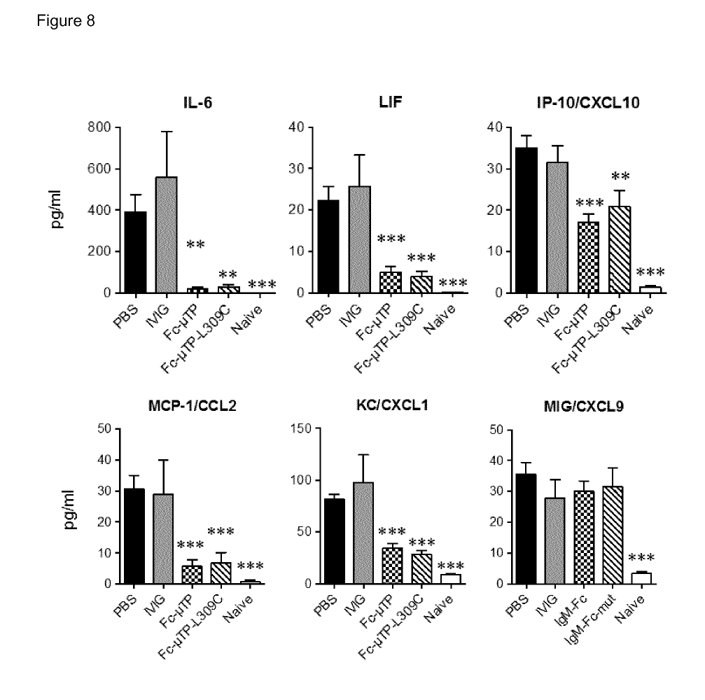

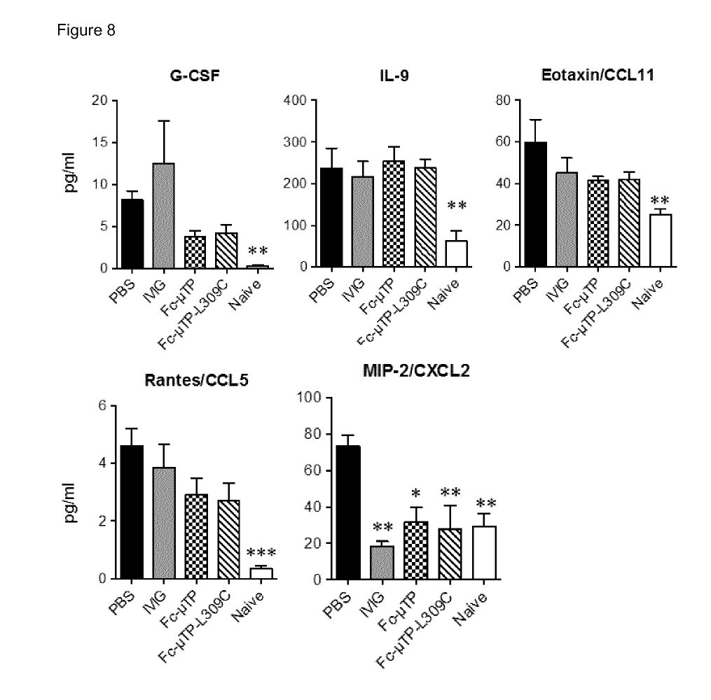

FIG. 1D shows the asymmetrical flow field-flow fractionation (AF4) of Fc-μTP (left) and Fc-μTP-L309C (right). Chromatograms show the normalized A280 signals and the thick bold lines show the molecular weight (in kDa) of material eluted at the time indicated, determined by MALS.

FIG. 2 shows activation of NFκB by LPS but not the Fc proteins Fc-μTP and Fc-μTP-L309C indicating the lack of endotoxin contamination.

FIG. 3A shows Biacore analysis of binding of Fc proteins Fc-μTP and Fc-μTP-L309C to FcγR components CD16a, CD32a, CD32b/c and CD64.

FIG. 3B shows binding of IVIG, Fc-μTP and Fc-μTP-L309C to the human monocytic cell line THP1.

FIG. 3C shows binding of IVIG, Fc, Fc-μTP and Fc-μTP-L309C to primary human neutrophils.

FIG. 3D shows immunofluorescence images, demonstrating binding of IVIG, Fc, Fc-μTP and Fc-μTP-L309C to primary human M1 and M2 macrophages.

FIG. 3E shows binding of IgG1, Fc, Fc-μTP and Fc-μTP-L309C to CD16-transfected NFAT-bla Jurkat cells. Ki values (mean±SEM, n=4 experiments) are shown in nM.

FIG. 4A shows representative flow cytometry histograms showing staining (grey shade) for (left to right) CD64 (FcγRI), CD32 (FcγRII) and CD16 (FcγRIII) on THP1 cells. Unfilled histograms show isotype control Ab staining.

FIG. 4B shows flow cytometry mean fluorescence intensity (MFI) values (mean±range, n=2) for (left to right) CD64, CD32 and CD16 on THP-1 cells.

FIG. 5A shows Octet analysis of binding of Fc, Fc-μTP and Fc-μTP-L309C (each at 25 pg/ml) to FcRn at pH 6.0.

FIG. 5B shows binding of human IgG1, Fc, Fc-μTP and Fc-μTP-L309C to FcRn-transfected FreeStyle™293-F cells at pH 5.5. Ki values (mean±SEM, n=3 experiments) are shown in nM.

FIG. 6A shows the pharmacokinetics of IVIG, Fc-μTP and Fc-μTP-L309C measured in the blood of FcRn-transgenic mice following a single i.v. injection at time 0. All doses were 100 mg/kg.

FIG. 6B shows the pharmacokinetics of IVIG, Fc-μTP and Fc-μTP-L309C measured in the blood of wild-type rats following a single i.v. injection at time 0. Doses were: IVIG (250 mg/kg), Fc-μTP (25 mg/kg), and Fc-μTP-L309C (25 mg/kg). The dashed horizontal line indicates the lower limit of detection in the assay.

FIG. 7A shows the protocol for evaluating the therapeutic effects of the Fc proteins in the CAbIA model of arthritis.

FIG. 7B shows the clinical response to therapeutic administration of IVIG, Fc-μTP and Fc-μTP-L309C at day 6 of the CAbIA model of arthritis. Kinetics of response (left) and mean clinical scores over days 7-14 (right) are shown. All data are means±SEM, pooled from 2 experiments.

FIG. 7C shows CD45+ cells recovered from knee joints of mice at day 8 of disease in the CAbIA model of arthritis. All data are means±SEM, pooled from 2 experiments. *P<0.05, **P<0.01, ***P<0.001, compared to PBS control.

FIG. 7D shows the histopathology of arthritic joints in the CAbIA model of arthritis. Representative H&E stained sections of tarsal joints from arthritic mice at day 8 that were treated at day 6 with either PBS, Fc-μTP or Fc-μTP-L309C. The joint of a naive non-arthritic mouse is also shown. Original magnification ×40.

FIG. 7E shows the histological analysis of joints in the CAbIA model of arthritis. Data show the mean (±SEM) histological scores of joints at days 8 and 14 from arthritic mice that were treated at day 6 with either PBS, Fc-μTP or Fc-μTP-L309C. All data are means±SEM, pooled from 2 experiments. *P<0.05, **P<0.01, ***P<0.001, compared to PBS control.

FIG. 8 shows cytokine/chemokine levels (in pg/ml) in joint tissue washes at day 8 of the CAbIA model of arthritis. All data are means±SEM, pooled from 2 experiments. *P<0.05, **P<0.01, ***P<0.001, compared to PBS control.

FIG. 9 shows complement components C1q, C3 and C5a in arthritic mouse joint washes taken at day 8 of the CAbIA model of arthritis. Arthritic mice were treated at day 6 with either PBS, Fc-μTP or Fc-μTP-L309C. Non-arthritic naive mouse joint washes were also included. Data (pooled from 2 experiments) show the complement component concentrations (means±SEM) in pg/ml, determined by ELISA. *P<0.05, **P<0.01, ***P<0.001, compared to PBS control.

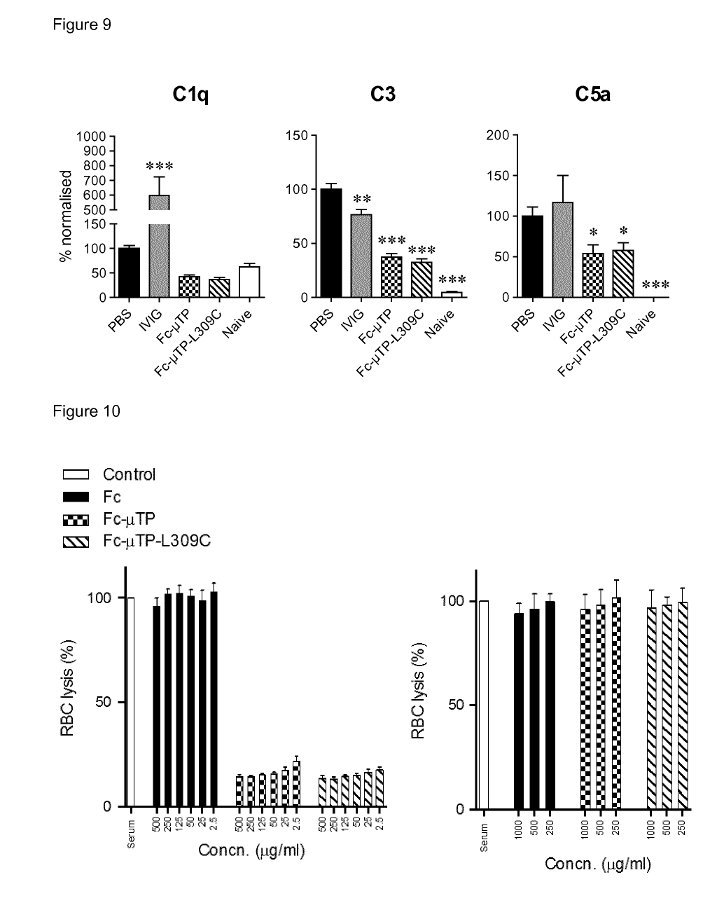

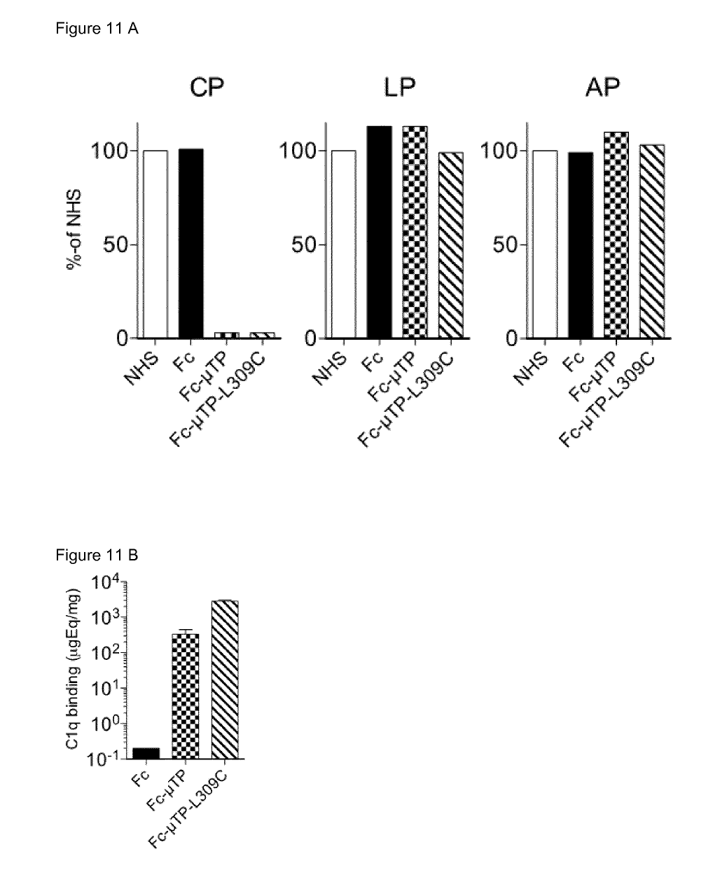

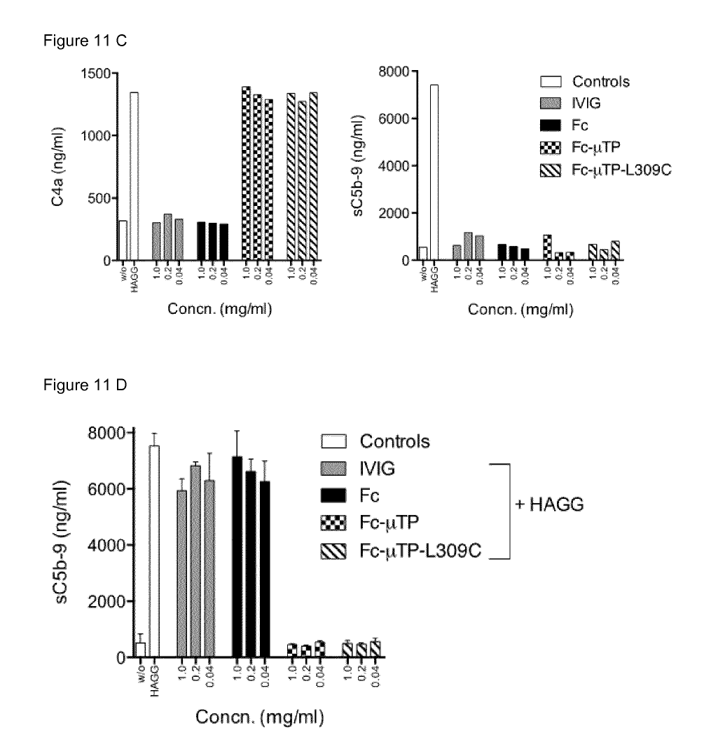

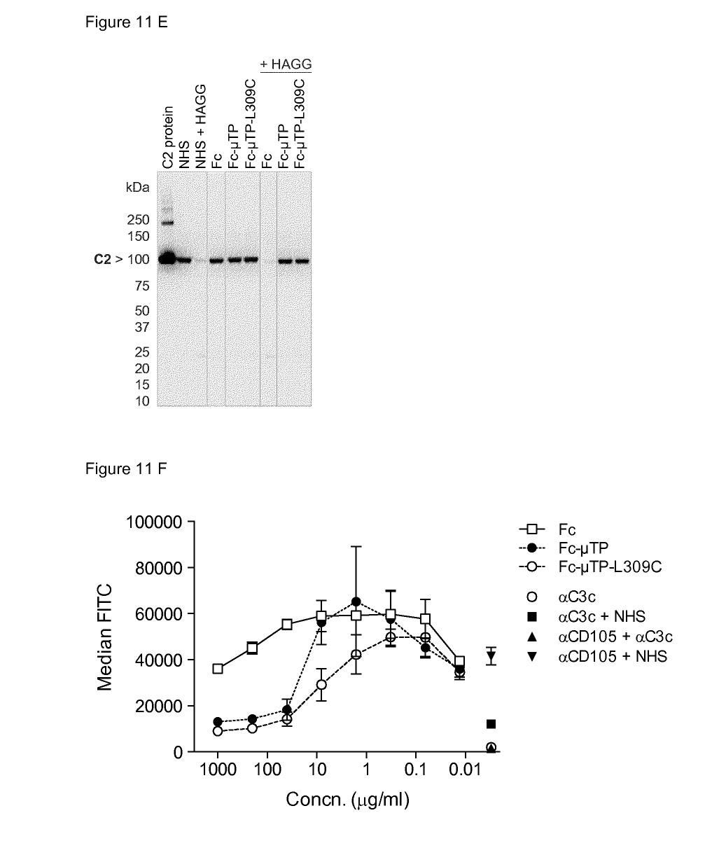

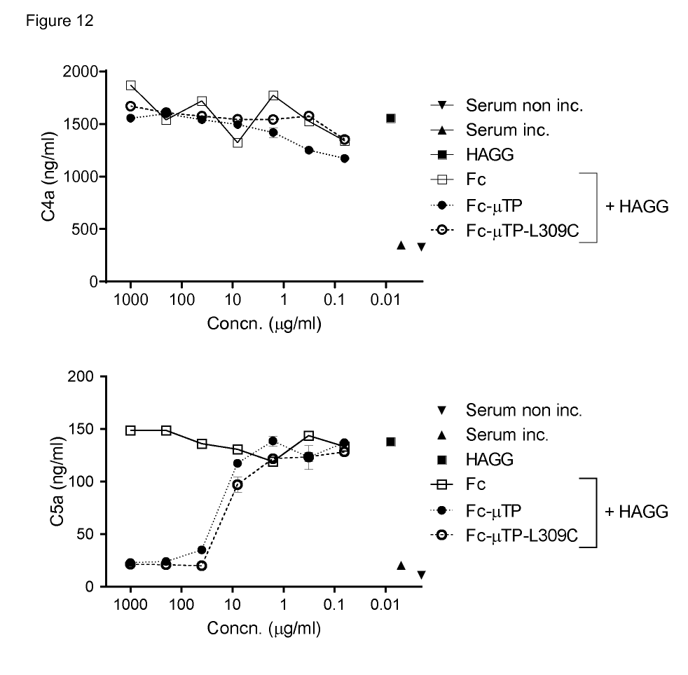

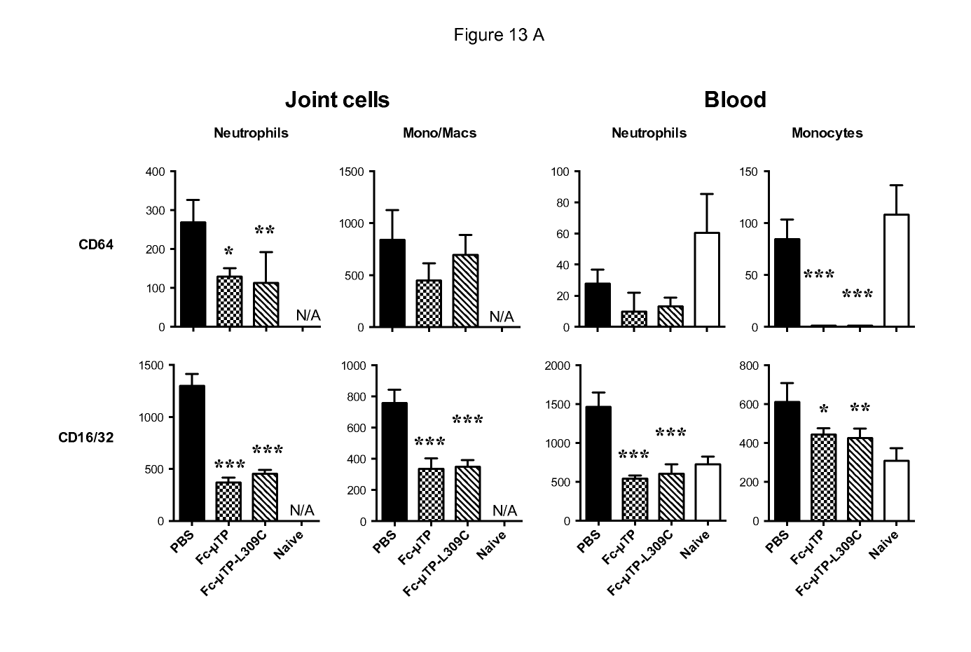

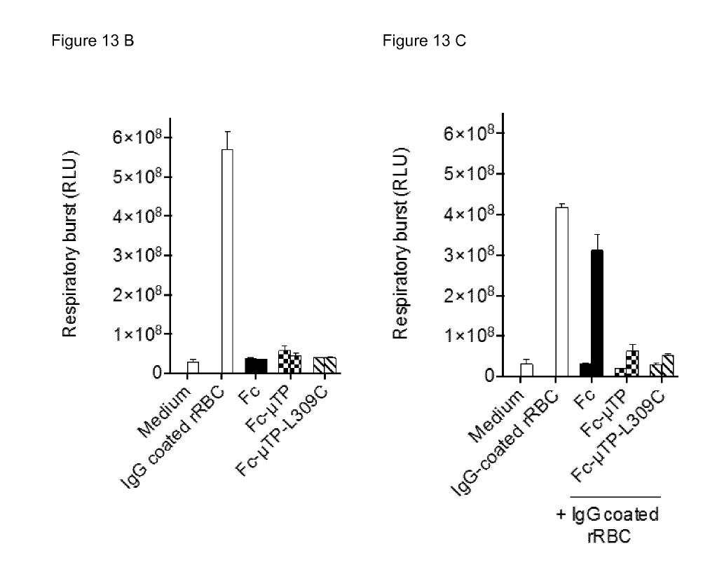

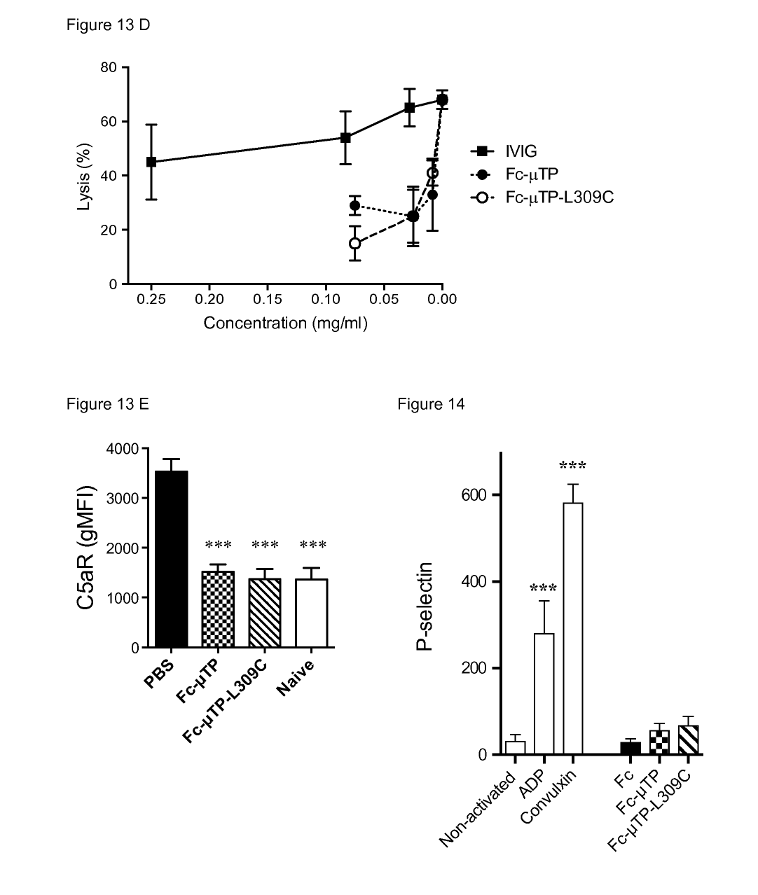

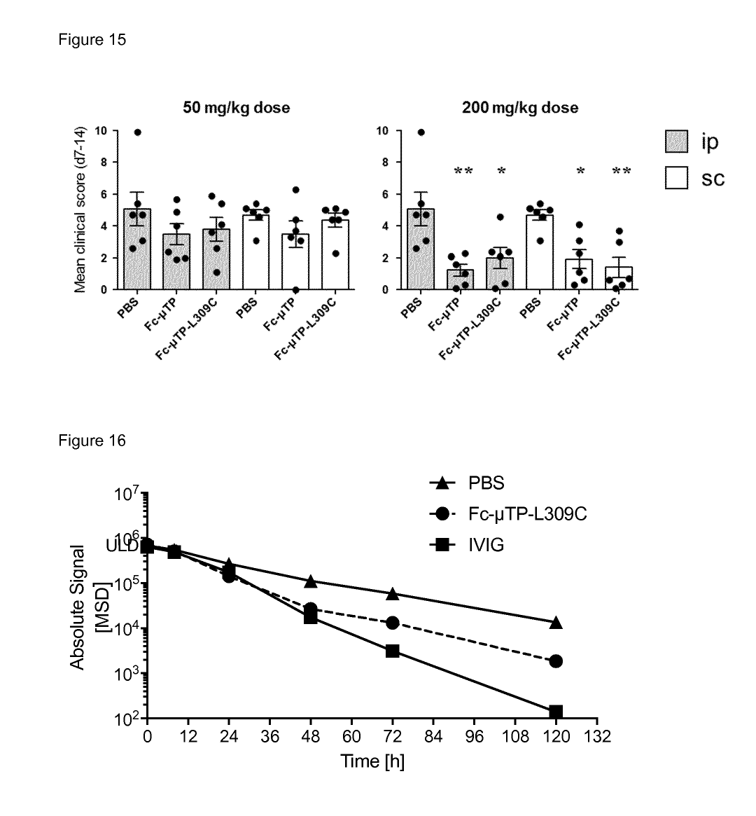

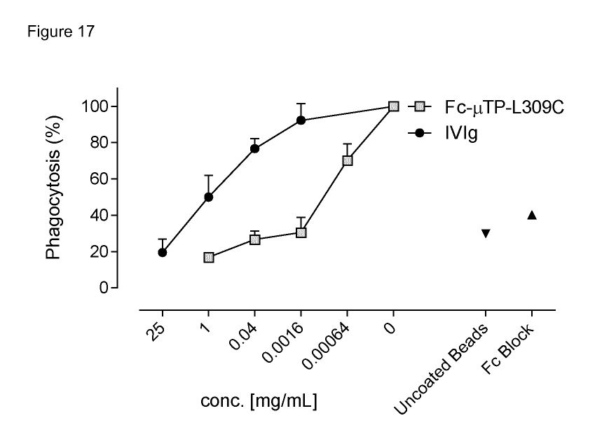

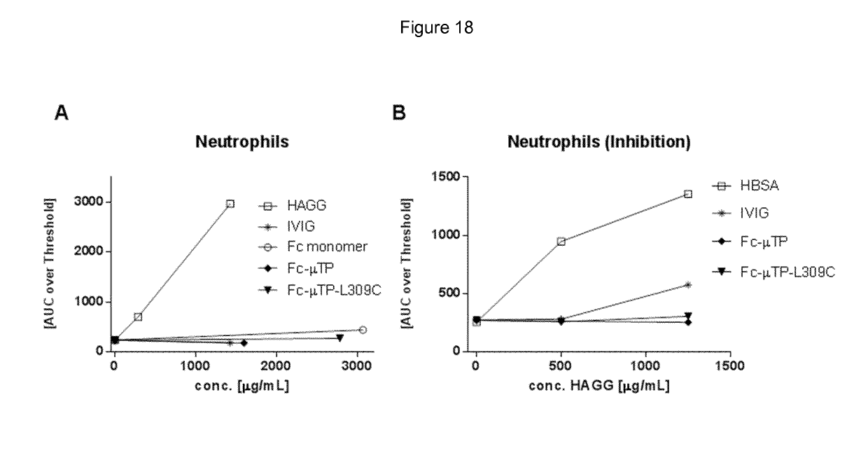

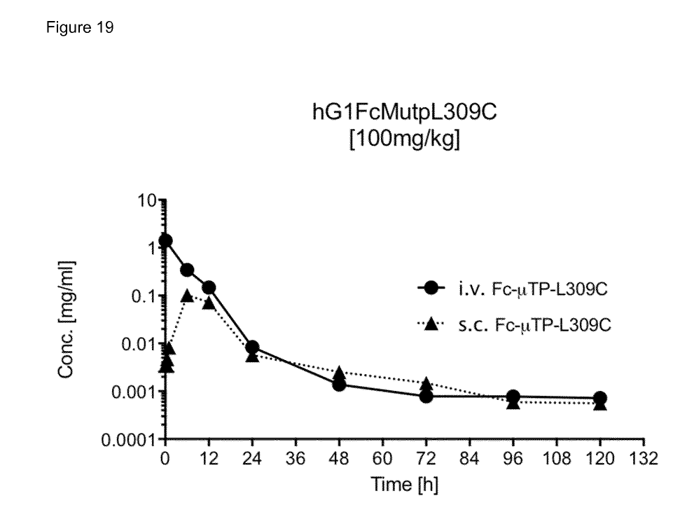

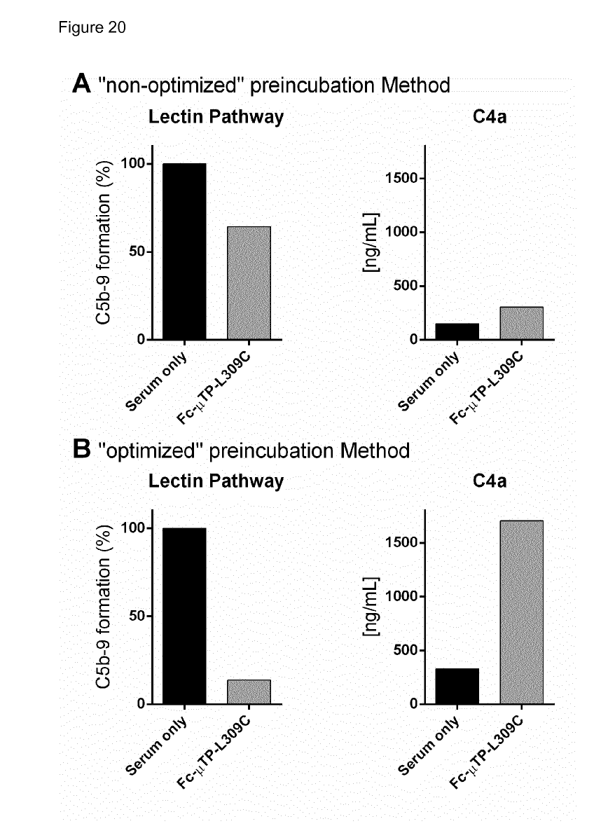

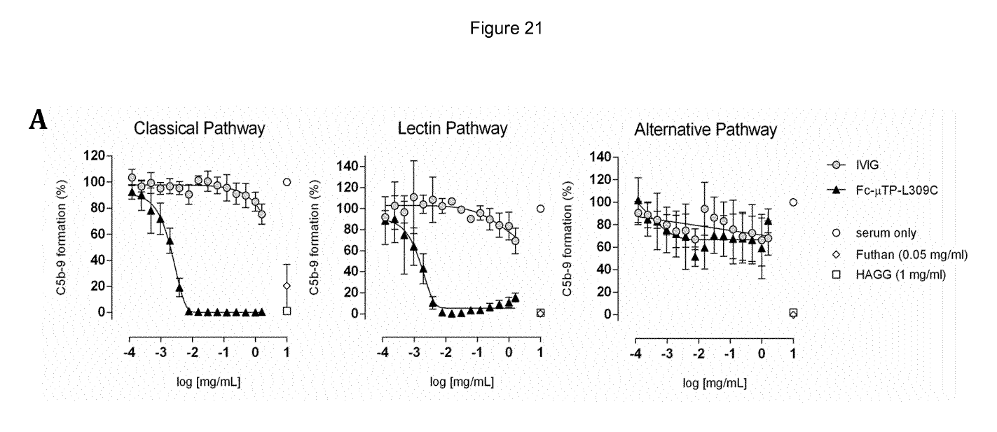

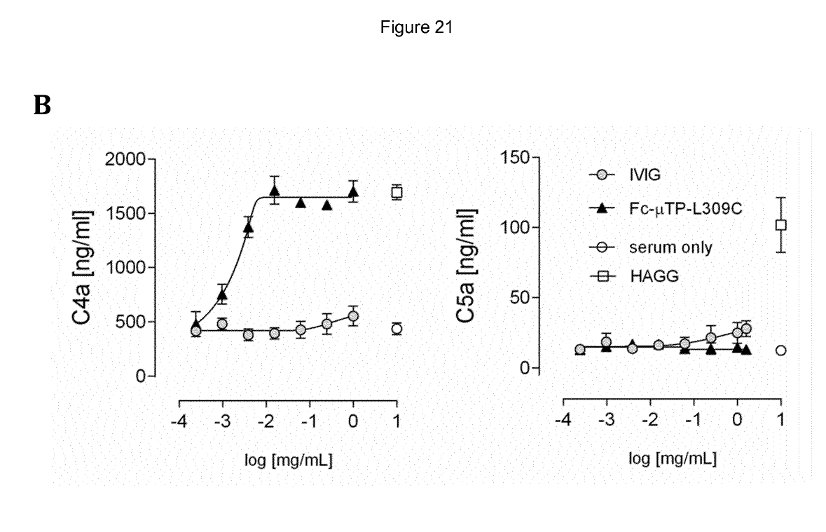

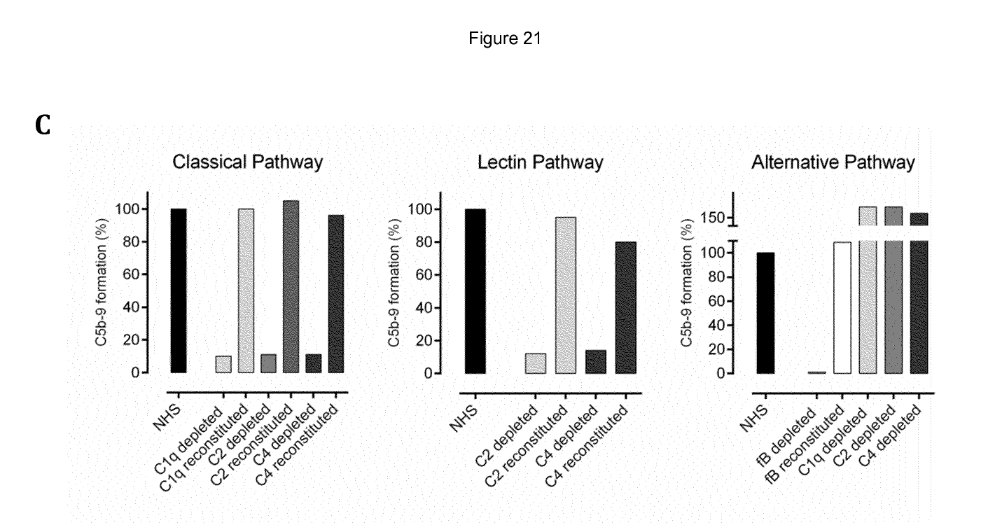

FIG. 10 shows the effect of Fc proteins Fc-μTP and Fc-μTP-L309C in hemolytic complement assays. Fc-μTP and Fc-μTP-L309C inhibit the classical pathway (lysis of opsonized sheep RBC; left panel) but not the alternative pathway (lysis of rabbit RBC; right panel).

FIG. 11A shows Inhibition using “non-optimized” buffer conditions of specific complement pathways (CP, classical; LP, lectin; AP, alternative) by Fc-μTP and Fc-μTP-L309C in vitro. Wieslab® ELISA Complement system screen kits were used. Data show the percentage of normal human serum (NHS) values.

FIG. 11B shows C1q binding to Fc, Fc-μTP or Fc-μTP-L309C, determined by ELISA.

FIG. 11C shows the effect of IVIG, Fc, Fc-μTP and Fc-μTP-L309C on the generation of C4a (left) and sC5b-9 (right) in human whole blood. Heat-aggregated gamma globulin (HAGG) served as a positive control.

FIG. 11D shows Fc-μTP and Fc-μTP-L309C inhibition of sC5b-9 generation in response to HAGG in human whole blood.

FIG. 11E shows effect of Fc-μTP and Fc-μTP-L309C on C2 cleavage in the presence and absence of HAGG, demonstrated by SDS-PAGE and Western blot for C2. The position of C2 is indicated by an arrow and molecular weight markers are shown at left in kDa.

FIG. 11F shows the dose-dependent inhibition of C3b deposition on HUVECs by Fc-μTP and Fc-μTP-L309C, not Fc monomer.

FIG. 12 shows the effect of Fc-μTP and Fc-μTP-L309C on the generation of C4a and C5a in HAGG-activated human serum.

FIG. 13A shows FcR (CD64, CD16/32) expression on neutrophils and monocytes obtained from the joints and blood of arthritic mice at day 8 of the CAbIA model of arthritis, that were treated at day 6 with either PBS, Fc-μTP or Fc-μTP-L309C. Data show the mean fluorescence intensity (MFI) (mean±SEM), determined by flow cytometry. *P<0.05, **P<0.01, ***P<0.001, compared to PBS control. N/A, not assessed.

FIG. 13B shows that Fc-μTP and Fc-μTP-L309C fail to activate the respiratory burst in human neutrophils. Left and right columns are 1.5 and 0.4 mg/ml doses, respectively.

FIG. 13C shows that Fc-μTP and Fc-μTP-L309C inhibit the activation of the respiratory burst in response to IgG-coated rabbit RBCs in human neutrophils. Left and right columns are 1.5 and 0.4 mg/ml doses, respectively.

FIG. 13D shows that Fc-μTP and Fc-μTP-L309C inhibit Ab-dependent cell-mediated cytotoxicity (ADCC) of anti-D-treated O+ human erythrocytes (mean±SEM, n=3).

FIG. 13E shows that Fc-μTP and Fc-μTP-L309C inhibit upregulation of C5aR (CD88) on peripheral blood monocytes at day 8 of the CAbIA model of arthritis.

FIG. 14 shows the lack of activation of human platelets by Fc-μTP and Fc-μTP-L309C. Data show P-selectin (CD62P) expression (MFI) on human platelets by flow cytometry as a marker of platelet activation. ADP or Convulxin served as a positive control for platelet activation. Data show the median fluorescence intensity (MFI) (mean±SEM; n=3), determined by flow cytometry. ***P<0.001, compared to non-activated.

FIG. 15 shows the clinical response to therapeutic administration of IVIG, Fc-μTP and Fc-μTP-L309C at day 6 of the CAbIA model of arthritis administered i.p. compared to s.c. Mean clinical scores over days 7-14 are shown. *P<0.05, **P<0.01, cf. PBS control; Kruskal-Wallis with Dunn's multiple comparison test.

FIG. 16 shows the effect of Fc-μTP-L309C, IVIG or PBS on the clearance of a tracer mAb in vivo in human FcRn transgenic mice. Data show the absolute signal of tracer mAb in serum of mice (mean±SEM; n=3) as determined by MSD-based immunoassay at a 1/500 dilution.

FIG. 17 shows the effect of Fc-μTP-L309C on the phagocytic activity of THP1 cells. THP1 cells were incubated with IgG coated FITC-labeled latexbeads and IVIG, Fc monomer or Fc-μTP-L309C for 3 hours at 37 degrees. Afterwards, uptake of beads was analyzed by flow cytometry. Data shown are normalized i.e. THP1 cells incubated only with IgG coated FITC-labeled latexbeads and medium were considered possessing 100% phagocytic activity (mean±SD, n=3).

FIG. 18A shows the effect of Fc-μTP-L309C on Ca²⁺ flux in primary neutrophils. Representative data shown.

FIG. 18B shows inhibition of HAGG induced Ca²⁺ mobilization with IVIG [2500 μg/mL], Fc-μTP [311 μg/mL] and Fc-μTP-L309C [274 μg/mL]. Representative data shown.

FIG. 19 shows bioavailability of Fc-μTP-L309C given i.v. versus s.c.

FIG. 20 shows effect of optimized preincubation method on inhibition of the lectin pathway of complement and C4 cleavage.

FIG. 21A shows inhibition using “optimized” buffer conditions of specific complement pathways (CP, classical; LP, lectin; AP, alternative) by Fc-μTP-L309C in vitro. Wieslab® ELISA Complement system kits (mean±SD, n=3).

FIG. 21B demonstrates generation of C4a and absence of C5a generation in normal human serum by Fc-μTP-L309C. As positive control heat aggregated IgG has been used (HAGG). (mean±SD, n=3).

FIG. 21C shows specificity of the Wieslab® ELISA Complement system kits using serum depleted of the indicated complement factors. Depleted sera were reconstituted with the respective purified protein to demonstrate complement activity of the used serum.

### DETAILED DESCRIPTION

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed as limiting.

The term “Fc monomer,” as used herein, is defined as a portion of an immunoglobulin G (IgG) heavy chain constant region containing the heavy chain CH2 and CH3 domains of IgG, or a variant or fragment thereof. The IgG CH2 and CH3 domains are also referred to as Cγ2 and Cγ3 domains respectively.

The Fc monomer may be comprised of two identical Fc peptides linked by disulfide bonds between cysteine residues in the N-terminal parts of the peptides. The arrangement of the disulfide linkages described for IgG pertain to natural human antibodies. There may be some variation among antibodies from other vertebrate species, although such antibodies may be suitable in the context of the present invention. The Fc peptides may be produced by recombinant expression techniques and associate by disulfide bonds as occurs in native antibodies. Alternatively, one or more new cysteine residues may be introduced in an appropriate position in the Fc peptide to enable disulfide bonds to form. Alternatively, Fc monomers may be comprised of two non-identical Fc peptides that form heterodimeric Fc monomers. In one possible approach, amino acid (aa) changes are performed to form two complementary Fc peptides. A small aa is replaced for a larger to form a “knob” in one Fc peptide and a large aa is replaced for a smaller to form a “hole” in the same region (e.g. CH3 domain) of another Fc peptide. With such a “knobs-into-holes” technology the self-assembly of two Fc peptides to form heterodimeric Fc monomers may be enhanced (Ridgway et al (1996) Protein Engineering 9: 617-621).

In one embodiment, the Fc monomer comprises two identical peptide chains comprising the human IgG1 CH2 and CH3 domains.

In another embodiment, the Fc monomer includes the entire CH2 and CH3 domains and is truncated at the N-terminus end of CH2 or the C-terminus end of CH3, respectively. Typically, the Fc monomer lacks the Fab polypeptide of the immunoglobulin. The Fab polypeptide is comprised of the CH1 domain and the heavy chain variable region domain.

The Fc monomer may comprise more than the CH2 and CH3 portion of an immunoglobulin. For example, in one embodiment, the monomer includes the hinge region of the immunoglobulin, a fragment or variant thereof, or a modified hinge region. A native hinge region is the region of the immunoglobulin which occurs between CH1 and CH2 domains in a native immunoglobulin. A variant or modified hinge region is any hinge that differs in length and/or composition from the native hinge region. Such hinges can include hinge regions from other species. Other modified hinge regions comprise a complete hinge region derived from an antibody of a different class or subclass from that of the Fc portion. Alternatively, the modified hinge region comprises part of a natural hinge or a repeating unit in which each unit in the repeat is derived from a natural hinge region. In another alternative, the natural hinge region is altered by increasing or decreasing the number of cysteine residues. Other modified hinge regions are entirely non-natural and are designed to possess desired properties such as length, cysteine composition, and flexibility.

A number of modified hinge regions have been described, for example in U.S. Pat. No. 5,677,425, WO 1999/15549, WO 2005/003170, WO 2005/003169, WO 2005/003170, WO 1998/25971, and WO 2005/003171 and these are incorporated herein by reference.

In one embodiment, the Fc peptide possesses a human IgG1 hinge region at its N-terminus. In one embodiment, the hinge region is SEQ ID NO:5.

In some embodiments, the Fc polypeptide chain comprises a signal peptide. The signal peptide directs the secretion of the Fc polypeptide chain and thereafter is cleaved from the remainder of the Fc polypeptide chain.

In one embodiment, the Fc peptide includes a signal peptide fused to the N-terminus of the hinge region. In one embodiment, the signal peptide is SEQ ID NO:4.

In order to improve formation of multimeric structures of two or more Fc monomers, the Fc peptide is fused to a tailpiece, which causes the monomer units to assemble into a multimer. The product of the fusion of the Fc peptide to the tailpiece is the “Fc fusion peptide,” as used herein. As Fc peptides dimerize to form Fc monomers, Fc fusion peptides likewise dimerize to form Fc fusion monomers.

An “Fc fusion monomer” as used herein therefore comprises two Fc fusion polypeptide chains and each Fc fusion polypeptide chain comprises an IgG Fc polypeptide and an IgM tailpiece.

Suitable tailpieces are derived from IgM or IgA. IgM and IgA occur naturally in humans as covalent multimers of the common H₂L₂ antibody unit. IgM occurs as a pentamer when it has incorporated a J chain, or as a hexamer when it lacks a J-chain. IgA occurs as monomers and forms dimers. The heavy chains of IgM and IgA each possess a respective 18 amino acid extension to the C-terminal constant domain, known as a tailpiece. This tailpiece includes a cysteine residue that forms a disulfide bond between heavy chains in the polymer, and is believed to have an important role in polymerization. The tailpiece also contains a glycosylation site.

The tailpiece of the present disclosure comprises any suitable amino acid sequence. The tailpiece is a tailpiece found in a naturally occurring antibody, or alternatively, it is a modified tailpiece which differs in length and/or composition from a natural tailpiece. Other modified tailpieces are entirely non-natural and are designed to possess desired properties for multimerization, such as length, flexibility, and cysteine composition.

In one embodiment, the tailpiece comprises all or part of the 18 amino acid sequence from human IgM (SEQ ID NO:6). In another embodiment, the tailpiece is a fragment or variant of the human IgM tailpiece.

In one embodiment, the tailpiece is fused directly to the C-terminus of the Fc peptide to form the Fc fusion peptide. Alternatively, the tailpiece is fused indirectly by means of an intervening amino acid sequence. For example, in one embodiment, a short linker sequence is provided between the tailpiece and the Fc peptide. A linker sequence may be between 1 and 20 amino acids in length.

Formation of multimeric structures may be further improved by mutating leucine 309 of the Fc portion of the Fc fusion peptide to cysteine. The L309C mutation allows for additional disulfide bond formation between the Fc fusion monomers, which further promotes multimerization of the Fc fusion monomers. The residues of the IgG Fc portion are numbered according to the EU numbering system for IgG, described in Edelman G M et al (1969), Proc Natl Acad Sci 63, 78-85; see also Kabat et al., 1983, Sequences of proteins of immunological interest, US Department of Health and Human Services, National Institutes of Health, Washington, D.C. Leu 309 of IgG corresponds by sequence homology to Cys 414 in CO domain of IgM and Cys 309 in the Cα2 domain of IgA.

Other mutations additionally, or alternatively, are introduced in the Fc fusion peptide to achieve desirable effects. The term “mutation,” as used herein, includes a substitution, addition, or deletion of one or more amino acids. In some embodiments, the Fc fusion peptide comprises up to 20, up to 10, up to 5, or up to 2 amino acid mutations.

In some embodiments, the mutations are conservative amino acid changes. The term “conservative amino acid changes,” as used herein, refers to the change of an amino acid to a different amino acid with similar biochemical properties, such as charge, hydrophobicity, structure, and/or size. In some embodiments, the Fc fusion peptide comprises up to 20, up to 10, up to 5, or up to 2 conservative amino acid changes. In one embodiment, the Fc fusion peptide comprises up to 5 conservative amino acid changes.

A conservative amino acid change includes a change amongst the following groups of residues: Val, Ile, Leu, Ala, Met; Asp, Glu; Asn, Gln; Ser, Thr, Gly, Ala; Lys, Arg, His; and Phe, Tyr, Trp.

A “variant,” when used herein to describe a peptide, protein, or fragment thereof, may have modified amino acids. Suitable modifications include acetylation, glycosylation, hydroxylation, methylation, nucleotidylation, phosphorylation, ADP-ribosylation, and other modifications known in the art. Such modifications may occur post-translationally where the peptide is made by recombinant techniques. Otherwise, modifications may be made to synthetic peptides using techniques known in the art. Modifications may be included prior to incorporation of an amino acid into a peptide. Carboxylic acid groups may be esterified or may be converted to an amide, an amino group may be alkylated, for example methylated. A variant may also be modified post-translationally, for example to remove or add carbohydrate side-chains or individual sugar moieties.

The term “Fc multimer,” as used herein, describes two or more polymerized Fc fusion monomers. An Fc multimer comprises two to six Fc fusion monomers, producing Fc dimers, Fc trimers, Fc tetramers, Fc pentamers, and Fc hexamers. Fc fusion monomers naturally associate into polymers having different numbers of monomer units.

In one embodiment, the majority of Fc multimer is an Fc hexamer. As used herein, the term “majority” refers to greater than 50%, greater than 60%, greater than 70%, greater than 80%, or greater than 90%. In one embodiment, greater than 80% of the Fc multimer is an Fc hexamer.

If Fc multimers containing a specific number of monomers are required, Fc multimers can be separated according to molecular size, for example by gel filtration (size exclusion chromatography).

### Polynucleotides

The disclosure further relates to a polynucleotide encoding an Fc fusion peptide for an Fc multimer. The term “polynucleotide(s)” generally refers to any polyribonucleotide or polydeoxyribonucleotide that may be unmodified RNA or DNA or modified RNA or DNA. The polynucleotide can be single- or double-stranded DNA, single or double-stranded RNA. As used herein, the term “polynucleotide(s)” also includes DNAs or RNAs that comprise one or more modified bases and/or unusual bases, such as inosine. It will be appreciated that a variety of modifications may be made to DNA and RNA that serve many useful purposes known to those of skill in the art. The term “polynucleotide(s)” as it is employed herein embraces such chemically, enzymatically, or metabolically modified forms of polynucleotides, as well as the chemical forms of DNA and RNA characteristic of viruses and cells, including, for example, simple and complex cells.

The skilled person would understand that, due to the degeneracy of the genetic code, a given polypeptide can be encoded by different polynucleotides. These “variants” are encompassed by the Fc multimers disclosed herein.

The polynucleotides of the Fc multimers may be an isolated polynucleotide. The term “isolated” polynucleotide refers to a polynucleotide that is substantially free from other nucleic acid sequences, such as and not limited to other chromosomal and extrachromosomal DNA and RNA. In one embodiment, the isolated polynucleotides are purified from a host cell. Conventional nucleic acid purification methods known to skilled artisans may be used to obtain isolated polynucleotides. The term also includes recombinant polynucleotides and chemically synthesized polynucleotides.

Another aspect of the disclosure is a plasmid or vector comprising a polynucleotide according to the disclosure. In one embodiment, the plasmid or vector comprises an expression vector. In one embodiment, the vector is a transfer vector for use in human gene therapy. Another aspect of the disclosure is a host cell comprising a polynucleotide, a plasmid, or vector of the disclosure.

In one embodiment, the host cell of the disclosure is employed in a method of producing an Fc multimer. The method comprises:
(a) culturing host cells of the disclosure under conditions such that the desired insertion protein is expressed; and
(b) optionally recovering the desired insertion protein from the host cells or from the culture medium.

In one embodiment, the Fc multimers are purified to ≥80% purity, ≥90% purity, ≥95% purity, ≥99% purity, or ≥99.9% purity with respect to contaminating macromolecules, for example other proteins and nucleic acids, and free of infectious and pyrogenic agents. An isolated Fc multimer of the disclosure may be substantially free of other, non-related polypeptides.

The various products of the disclosure are useful as medicaments. Accordingly, the disclosure relates to a pharmaceutical composition comprising an Fc multimer, a polynucleotide of the disclosure, or a plasmid or vector of the disclosure.

The disclosure also concerns a method of treating an autoimmune or inflammatory disease in a subject in need thereof. The method comprises administering to said subject a therapeutically effective amount of the Fc multimer. In another embodiment, the method comprises administering to said subject a therapeutically effective amount of a polynucleotide of the disclosure or a plasmid or vector of the disclosure.

### Expression of the Proposed Fc Multimers

The production of recombinant proteins at high levels in suitable host cells requires the assembly of the above-mentioned modified cDNAs into efficient transcriptional units together with suitable regulatory elements in a recombinant expression vector that can be propagated in various expression systems according to methods known to those skilled in the art. Efficient transcriptional regulatory elements could be derived from viruses having animal cells as their natural hosts or from the chromosomal DNA of animal cells. For example, promoter-enhancer combinations derived from the Simian Virus 40, adenovirus, BK polyoma virus, human cytomegalovirus, or the long terminal repeat of Rous sarcoma virus, or promoter-enhancer combinations including strongly constitutively transcribed genes in animal cells like beta-actin or GRP78 can be used. In order to achieve stable high levels of mRNA transcribed from the cDNAs, the transcriptional unit should contain in its 3′-proximal part a DNA region encoding a transcriptional termination-polyadenylation sequence. For example, this sequence can be derived from the Simian Virus 40 early transcriptional region, the rabbit beta globin gene, or the human tissue plasminogen activator gene.

The cDNAs can then be integrated into the genome of a suitable host cell line for expression of the Fc multimer. In some embodiments, this cell line should be an animal cell-line of vertebrate origin in order to ensure correct folding, disulfide bond formation, asparagine-linked glycosylation and other post-translational modifications as well as secretion into the cultivation medium. Examples of other post-translational modifications are tyrosine 0-sulfation and proteolytic processing of the nascent polypeptide chain. Examples of cell lines that can be used are monkey COS-cells, mouse L-cells, mouse C127-cells, hamster BHK-21 cells, human embryonic kidney 293 cells, and hamster CHO-cells.

The recombinant expression vector encoding the corresponding cDNAs can be introduced into an animal cell line in several different ways. For example, recombinant expression vectors can be created from vectors based on different animal viruses. Examples of these are vectors based on baculovirus, vaccinia virus, adenovirus, and bovine papilloma virus.

The transcription units encoding the corresponding DNAs can also be introduced into animal cells together with another recombinant gene which may function as a dominant selectable marker in these cells in order to facilitate the isolation of specific cell clones which have integrated the recombinant DNA into their genome. Examples of this type of dominant selectable marker genes are TN4 amino glycoside phosphotransferase, conferring resistance to geneticin (G418), hygromycin phosphotransferase, conferring resistance to hygromycin, and puromycin acetyl transferase, conferring resistance to puromycin. The recombinant expression vector encoding such a selectable marker can reside either on the same vector as the one encoding the cDNA of the desired protein, or it can be encoded on a separate vector which is simultaneously introduced and integrated to the genome of the host cell, frequently resulting in a tight physical linkage between the different transcription units.

Other types of selectable marker genes which can be used together with the cDNA of the desired protein are based on various transcription units encoding dihydrofolate reductase (dhfr). After introduction of this type of gene into cells lacking endogenous dhfr-activity, for example CHO-cells (DUKX-B11, DG-44), it will enable these to grow in media lacking nucleosides. An example of such a medium is Ham's F12 without hypoxanthine, thymidine, and glycine. These dhfr-genes can be introduced together with the cDNA encoding the IgG Fc fusion monomer into CHO-cells of the above type, either linked on the same vector on different vectors, thus creating dhfr-positive cell lines producing recombinant protein.

If the above cell lines are grown in the presence of the cytotoxic dhfr-inhibitor methotrexate, the new cell lines resistant to methotrexate will emerge. These cell lines may produce recombinant protein of at an increased rate due to the amplified number of linked dhfr and the desired protein's transcriptional units. When propagating these cell lines in increasing concentrations of methotrexate (1-10,000 nM), new cell lines can be obtained which produce the desired protein at very high rate.

The above cell lines producing the desired protein can be grown on a large scale, either in suspension culture or on various solid supports. Examples of these supports are micro carriers based on dextran or collagen matrices, or solid supports in the form of hollow fibers or various ceramic materials. When grown in cell suspension culture or on micro carriers the culture of the above cell lines can be performed either as a bath culture or as a perfusion culture with continuous production of conditioned medium over extended periods of time. Thus, according to the present disclosure, the above cell lines are well suited for the development of an industrial process for the production of the desired recombinant proteins.

### Purification and Formulation

The recombinant protein can be concentrated and purified by a variety of biochemical and chromatographic methods, including methods utilizing differences in size, charge, hydrophobicity, solubility, specific affinity, etc., between the desired protein and other substances in the host cell or cell cultivation medium.

An example of such purification is the adsorption of the recombinant protein to a monoclonal antibody directed to e.g. the Fc portion of the Fc multimer or another Fc-binding ligand (e.g. protein A or protein G), which is immobilized on a solid support. After adsorption of the Fc multimer to the support, washing and desorption, the protein can be further purified by a variety of chromatographic techniques based on the above properties. The order of the purification steps is chosen, for example, according to capacity and selectivity of the steps, stability of the support or other aspects. Purification steps, for example, may be, but are not limited to, ion exchange chromatography steps, immune affinity chromatography steps, affinity chromatography steps, dye chromatography steps, and size exclusion chromatography steps.

In order to minimize the theoretical risk of virus contaminations, additional steps may be included in the process that allow effective inactivation or elimination of viruses. For example, such steps may include heat treatment in the liquid or solid state, treatment with solvents and/or detergents, radiation in the visible or UV spectrum, gamma-radiation, partitioning during the purification, or virus filtration (nano filtration).

The Fc multimers described herein can be formulated into pharmaceutical preparations for therapeutic use. The components of the pharmaceutical preparation may be resuspended or dissolved in conventional physiologically compatible aqueous buffer solutions to which there may be added, optionally, pharmaceutical excipients to provide the pharmaceutical preparation. The components of the pharmaceutical preparation may already contain all necessary pharmaceutical, physiologically compatible excipients and may be dissolved in water for injection to provide the pharmaceutical preparation.

Such pharmaceutical carriers and excipients as well as the preparation of suitable pharmaceutical formulations are well known in the art (see for example, “Pharmaceutical Formulation Development of Peptides and Proteins,” Frokjaer et al., Taylor & Francis (2000) or “Handbook of Pharmaceutical Excipients,” 3rd edition, Kibbe et al., Pharmaceutical Press (2000)). In certain embodiments, a pharmaceutical composition can comprise at least one additive such as a bulking agent, buffer, or stabilizer. Standard pharmaceutical formulation techniques are well known to persons skilled in the art (see, e.g., 2005 Physicians' Desk Reference®, Thomson Healthcare: Monvale, N J, 2004; Remington: The Science and Practice of Pharmacy, 20th ed., Gennaro et al., Eds. Lippincott Williams & Wilkins: Philadelphia, Pa., 2000). Suitable pharmaceutical additives include, e.g., sugars like mannitol, sorbitol, lactose, sucrose, trehalose, or others, amino acids like histidine, arginine, lysine, glycine, alanine, leucine, serine, threonine, glutamic acid, aspartic acid, glutamine, asparagine, phenylalanine, proline, or others, additives to achieve isotonic conditions like sodium chloride or other salts, stabilizers like Polysorbate 80, Polysorbate 20, Polyethylene glycol, propylene glycol, calcium chloride, or others, physiological pH buffering agents like Tris(hydroxymethylaminomethan), and the like. In certain embodiments, the pharmaceutical compositions may contain pH buffering reagents and wetting or emulsifying agents. In further embodiments, the compositions may contain preservatives or stabilizers. In particular, the pharmaceutical preparation comprising the Fc multimers described herein may be formulated in lyophilized or stable soluble form. The Fc multimers factor may be lyophilized by a variety of procedures known in the art. Lyophilized formulations are reconstituted prior to use by the addition of one or more pharmaceutically acceptable diluents such as sterile water for injection or sterile physiological saline solution or a suitable buffer solution.

The composition(s) of the pharmaceutical preparation of Fc multimer may be delivered to the individual by any pharmaceutically suitable means. Various delivery systems are known and can be used to administer the composition by any convenient route. The composition(s) of the pharmaceutical preparation of the Fc multimer can be formulated for intravenous or non-intravenous injection or for enteral (e.g., oral, vaginal, or rectal) delivery according to conventional methods. For non-intravenous administration, the composition(s) of the Fc multimer can be formulated for subcutaneous, intramuscular, intra-articular, intraperitoneal, intracerebral, intrathecal, intrapulmonary (e.g. nebulized), intranasal, intradermal, peroral or transdermal administration. In one embodiment, the composition(s) of the Fc multimer are formulated for intravenous injection. In other embodiments, the composition(s) of the Fc multimer are formulated for subcutaneous, intramuscular, or transdermal administration, preferably for subcutaneous administration. The formulations can be administered continuously by infusion or by bolus injection. Some formulations can encompass slow release systems.

The composition(s) of the pharmaceutical preparation of Fc multimer is/are administered to patients in a therapeutically effective dose. The term “therapeutically effective,” as used herein, describes a dose that is sufficient to produce the desired effects, preventing or lessening the severity or spread of the condition or indication being treated, or to exhibit a detectable therapeutic or preventative effect, without teaching a dose which produces intolerable adverse side effects. The exact dose depends on many factors as, for example, the indication, formulation, and mode of administration. The therapeutically effective amount can be initially estimated in cell culture assays or in animal models, for example rodent, rabbit, dog, pig, or primate models. Such information can then be used to determine useful doses and routes for administration in humans.

In one embodiment, the dose of the Fc multimer for one intravenous or one non-intravenous injection is less than 1,000 mg/kg body weight, less than 800 mg/kg body weight, less than 600 mg/kg body weight, less than 400 mg/kg body weight, less than 200 mg/kg body weight, or less than 100 mg/kg body weight. For example, in one embodiment, the dose of Fc multimer is from about 1 mg/kg body weight to about 1,000 mg/kg body weight, from about 10 mg/kg body weight to about 800 mg/kg body weight, from about 20 mg/kg body weight to about 700 mg/kg body weight, from about 30 mg/kg body weight to about 600 mg/kg body weight, from about 40 mg/kg body weight to about 500 mg/kg body weight, from about 50 mg/kg body weight to about 400 mg/kg body weight, from about 75 mg/kg body weight to about 300 mg/kg body weight, or from about 100 mg/kg body weight to about 200 mg/kg body weight. In one embodiment, the dose of Fc multimer is from about 25 mg/kg body weight to about 1,000 mg/kg body weight, from about 25 mg/kg body weight to about 800 mg/kg body weight, from about 25 mg/kg body weight to about 600 mg/kg body weight, from about 25 mg/kg body weight to about 500 mg/kg body weight, from about 25 mg/kg body weight to about 400 mg/kg body weight, from about 25 mg/kg body weight to about 300 mg/kg body weight, from about 25 mg/kg body weight to about 200 mg/kg body weight, or from about 25 mg/kg body weight to about 100 mg/kg body weight.

In one embodiment, the pharmaceutical composition(s) of Fc multimer is administered alone or in conjunction with other therapeutic agents. In one embodiment, these agents are incorporated as part of the same pharmaceutical. In one embodiment, the Fc multimer is administered in conjunction with an immunosuppressant therapy, such as a steroid. In another embodiment, the Fc multimer is administered with any B cell or T cell modulating agent or immunomodulator.

The administration frequency of the Fc multimer depends on many factors such as the indication, formulation, dosage, and mode of administration. In one embodiment, a dose of Fc multimer is administered multiple times every day, once every day, once every other day, once every third day, twice per week, once per week, once every two weeks, once every three weeks, or once per month.

In one embodiment, Fc multimers of the present disclosure are used to treat autoimmune disease or inflammatory disease. “Autoimmune disease,” as used herein, includes any disease in which the immune system attacks the body's own tissues. “Inflammatory disease,” as used herein, includes any disease characterized by destructive inflammation which may be recurrent or chronic and is not associated with normal tissue repair. Such diseases particularly include “autoinflammatory diseases” in which the innate immune system causes inflammation for reasons which may be unknown. In one embodiment, Fc multimers of the present disclosure are used to treat auto-antibody mediated autoimmune disease. In one embodiment, the Fc multimers of the present disclosure are used to treat complement-mediated inflammation in transplantation. In one embodiment, the Fc multimers of the present disclosure are used to treat complement-mediated inflammation in reperfusion injury. In one embodiment, the Fc multimers of the present disclosure are used to treat complement-mediated inflammation in spinal cord injury.

Autoimmune or inflammatory diseases suitable for treatment include autoimmune cytopenia, idiopathic thrombocytopenic purpura/immune cytopenia (ITP), rheumatoid arthritis, systemic lupus erythematosus, asthma, Kawasaki disease, Guillain-Barré syndrome, Stevens-Johnson syndrome, Crohn's colitis, diabetes, chronic inflammatory demyelinating polyneuropathy, inflammatory neuropathy, neuromyelitis optica, other autoimmune channelopathies, autoimmune epilepsy, myasthenia gravis, anti-Factor VIII autoimmune disease, dermatomyositis, polymyositis, scleroderma, vasculitis, uveitis, pemphigus, pemphigoid, spinal cord injury or Alzheimer's disease.

### Therapeutic Effects

In some embodiments, the Fc multimer provides therapeutic effects. The term “therapeutic effects,” as used herein, describes improvements in parameters that characterize the disease or disorder. For example, therapeutic effects can be determined in animal models of diseases or disorders by administering a dose of an Fc multimer. A dose of Fc multimer can be 10 to 1000 mg/kg, for example, 200 mg/kg. The Fc multimer can be administered by intravenous or non-intravenous injection or intravenous infusion. Clinical assessments of animals can be made at predetermined times until a final time point after administration of the Fc multimer. Clinical assessments can include scoring based on clinical manifestations of the specific disease or disorder. Biological samples can also be taken from the animals at predetermined times until a final time point after administration of the Fc multimer. The term “biological samples,” as used herein, refers to, for example, tissue, blood, and urine. The biological samples can then be assessed for improvements in markers or indicators of the specific disease or disorder. Diseases or disorders for which therapeutic effects can be determined include autoimmune or inflammatory diseases.

In one embodiment, the animal model of disease is a model of arthritis. For example, the animal model of disease can be the anti-collagen antibody-induced arthritis (CAbIA) model in mice. In this model, arthritis can be induced in mice by intraperitoneal injection of a mouse monoclonal anti-type II collagen 5 clone antibody on day 0 followed by an injection of lipopolysaccharide on day 3. Clinical signs of arthritis in the mice can be assessed and scored on each day. For example, the following clinical scores can be used: 0—normal; 0.5—swelling confined to digits; 1—mild paw swelling; 2—marked paw swelling; 3—severe paw swelling and/or ankylosis. Scores for each paw of an individual animal can be summed for a total clinical score. The therapeutic effect of an Fc multimer can then be assessed by administering a dose of Fc multimer on day 6 to mice that have a clinical score of 1 or more, determining the clinical scores of the mice at predetermined times afterward, and comparing the clinical scores to clinical scores for mice that are untreated or administered a vehicle control, such as PBS. For example, clinical scores can be determined on days 7 through 14. A mean clinical score can be determined by averaging the clinical scores from days 7 through 14. A dose of Fc multimer can be 25 to 1000 mg/kg. In one embodiment, the dose of Fc multimer is 200 mg/kg. For example, the histological score can be determined as described in Example 5.

The term “mouse anti-collagen antibody-induced arthritis model” as used herein refers to the mouse arthritis model as described by Campbell, I K et al., (2014). J Immunol. 192(11):5031-8.

The term “day 6” as used herein in a mouse anti-collagen antibody-induced arthritis model refers to the 6th day after the injection of a monoclonal antibody inducing arthritis as described by Campbell, I K et al., (2014). J Immunol. 192(11):5031-8.

The term “arthritic mice” as used herein refers to mice subjected to an arthritis inducing monoclonal antibody as described by Campbell, I K et al., (2014). J Immunol. 192(11):5031-8.

The term “induce,” as used herein, is defined as to cause, produce, effect, create, give rise to, lead to, or promote.

A therapeutic effect of the Fc multimer can be indicated by a reduced clinical score in mice administered the Fc multimer compared to mice administered a vehicle control or untreated mice. In some embodiments, the Fc multimer induces a reduction in the clinical score at any of days 7 to 14 or in the mean clinical score from days 7 to 14 compared to untreated mice, or control mice administered PBS. In one embodiment, the Fc multimer induces a greater than 50% reduction in the clinical score at any of days 7 to 14 or a greater than 50 reduction in the mean clinical score from days 7 to 14 compared to untreated mice, or control mice administered PBS.

A therapeutic effect of the Fc multimer in the collagen antibody-induced arthritis (CAbIA) mouse model described above can also be determined by assessing the number of infiltrating cells in joints of the mice at a predetermined time after administering the Fc multimer. For example, at day 8 joints of the arthritic mice can be removed and processed. For example, patellas and surrounding soft tissue, excluding fat, can be removed and placed in standard medium on ice for 60 min. The medium can then be removed and centrifuged and the supernatant, or joint wash, can be stored for later analysis. The patellas and cell pellets can be combined and digested with collagenase and DNAse and then strained with a 70 μm cutoff, washed, and resuspended in buffer. The resuspended cells can then be stained with labeled antibodies. For example, cells can be stained with a labeled anti-CD45 antibody. CD45+ cells are indicative of infiltrating leukocytes. The number of CD45+ cells in mice can then be counted using flow cytometry. For example, the number of infiltrating CD45+ cells can be determined as described in Example 5.

A therapeutic effect can be indicated by a reduced number of CD45+ cells from joints of mice administered an Fc multimer compared to the number of CD45+ cells from joints of untreated mice, or mice administered a vehicle control, such as PBS. In some embodiments the Fc multimer induces a reduction in the number of CD45+ cells recovered from knee joints compared to untreated mice, or control mice administered PBS. In one embodiment, the Fc multimer induces a greater than 50% reduction in the number of CD45+ cells recovered from knee joints on day 8 compared to untreated mice, or control mice administered PBS.

A therapeutic effect of the Fc multimer in the collagen antibody-induced arthritis (CAbIA) mouse model described above can also be determined by assessing the histological score of the joints of the mice at a predetermined time after administering the Fc multimer. The predetermined time can be, for example, day 8 or day 14. For example, paws of arthritic mice can be removed, fixed in formalin, decalcified, and embedded in paraffin. Tissue sections can then be stained with hematoxylin and eosin (H&E) and the histopathology can be scored. For example, the following histopathological features can be scored: exudate—presence of inflammatory cells within the joint space; synovitis—the degree of synovial membrane thickening and inflammatory cell infiltration; tissue destruction—cartilage and bone erosion and invasion. Each feature can then be scored according to the following scale: 0—normal, 1—minimum, 2—mild, 3—moderate, 4—marked, 5—severe. The cumulative score for the three histopathological features can then be tallied. For example, the histological score can be determined as described in Example 5.

A therapeutic effect of the Fc multimer can be indicated by a reduced histological score in mice administered the Fc multimer compared to mice administered a vehicle control or untreated mice. In some embodiments the Fc multimer induces a reduction in the histological score at day 8, day 14, or both day 8 and day 14, compared to untreated mice, or control mice administered PBS. In one embodiment, the Fc multimer induces a greater than 25% reduction in the histological score at day 8 compared to untreated mice, or control mice administered PBS. In another embodiment, the Fc multimer induces a greater than 50% reduction in the histological score at day 14 compared to untreated mice, or control mice administered PBS.

### Reduction of Expression of FcγRs

There are three classes of human Fcγ receptor (Gessner et al (1998) Ann Hematol 76: 231-48; Raghavan and Bjorkman (1996) Ann Rev Cell Dev Biol 12: 181-220). FcγRI (CD64) binds monomeric IgG with high affinity. Human FcγRI on cells is normally considered to be ‘occupied’ by monomeric IgG in normal serum conditions due to its affinity for IgG1>>IgG2/IgG3/IgG4 (˜10⁻⁸) and the total concentration of these IgG in serum (˜10 mg/ml). As such, cells bearing FcγRI on their surface are considered to be capable for ‘screening’ or ‘sampling’ of their antigenic environment vicariously through the bound polyspecific IgG. FcγII (CD32) and FcγRIII (CD16) are the low affinity receptors (in the range of ˜10⁻⁵-10⁻⁷ M) and are normally considered to be ‘unoccupied.’ The low affinity receptors are hence inherently sensitive to the detection of and activation by antibody involved immune complexes.

Many cell types express multiple types of FcγR and so binding of IgG or antibody immune complex to cells bearing FcγR can have multiple complex outcomes depending on the biological context. For example, cells can either receive an activatory, inhibitory, or mixed signal. This can result in events such as phagocytosis (e.g. macrophages and neutrophils), antigen processing (e.g. dendritic cells), reduced IgG production (e.g. B-cells), or degranulation (e.g. neutrophils and mast cells).

In some embodiments, the Fc multimer reduces expression of FcγRs on neutrophils, monocytes, or macrophages in a subject with a disease or disorder, for example an autoimmune or inflammatory disease. The ability of an Fc multimer to reduce expression of FcγRs can be investigated in an animal model of disease, such as the collagen antibody-induced arthritis (CAbIA) mouse model described above. The expression of FcγRs on neutrophils, monocytes, or macrophages can be determined in joints and blood of the mice at a predetermined time after administering the Fc multimer. For example, at day 8 joints of the arthritic mice can be removed and processed as described above. Peripheral blood samples can also be obtained from the mice. Joint digests and peripheral blood can be resuspended in buffer and then stained with labelled antibodies directed to specific FcγRs, such as FcγRII/FcγRIII (CD32/16) or FcγRI (CD64), and stained cells can be assessed for FcγR expression by flow cytometry. FcγR expression on monocytes/macrophages and neutrophils from the joints and peripheral blood of mice administered an Fc multimer can be compared to FcγR on monocytes/macrophages and neutrophils untreated mice, for example mice administered PBS. For example, relative FcγR expression on monocytes/macrophages and neutrophils can be determined as described in Example 9.

In some embodiments, the Fc multimer reduces the expression of FcγRII/FcγRIII (CD32/16) on joint neutrophils, blood neutrophils, joint monocytes/macrophages, and/or blood monocytes/macrophages at day 8 of the CAbIA model compared to untreated, or PBS-administered, arthritic mice. In one embodiment, the Fc multimer reduces the expression of FcγRII/FcγRIII (CD32/16) on joint neutrophils, blood neutrophils, and joint monocytes/macrophages by greater than 50%, and reduces the expression of FcγRII/FcγRIII (CD32/16) on blood monocyte/macrophages by greater than 25% compared to mice administered PBS. In some embodiments the Fc multimer reduces the expression of FcγRI (CD64) on joint neutrophils and blood monocytes/macrophages. In one embodiment, the Fc multimer reduces the expression of FcγRI (CD64) on joint neutrophils by greater than 50% and the expression of FcγRI (CD64) on blood monocytes/macrophages by greater than 75% compared to arthritic mice administered PBS.

### Activation of the Classical Complement Pathway

The classical complement pathway mediates the specific antibody response and is mediated by a cascade of complement components. The cascade is mainly activated by antigen-antibody complexes. The initial component of the pathway is the protein complex C1, which is comprised of one C1q and two subunits of C1r2s2. Binding of an immunoglobulin to C1q effects the first step of activation of the classical complement pathway through activation of C1r2s2 into catalytically active subunits. The activated C1s cleaves C4 into C4a and C4b and C2 into C2a and C2b. C2a then binds C4b to form C4b2a, which is also known as C3 convertase. C3 convertase catalyzes the cleavage of C3 into C3a and C3b. C3b can then bind to activated C4b2a to form C4b2a3b, which is also known as C5 convertase. C5 convertase converts C5 to fragments C5a and C5b. C5b, together with the C6, C7, C8, and C9 components, forms a complex known as the C5b-9 complex. This complex is also known as the membrane attack complex (MAC) or terminal complement complex (TCC) and forms transmembrane channels in target cells, leading to cell lysis.

“Activation of the complete classical complement pathway”, as used herein, is defined as the activation of every step of the entire classical complement pathway as described above. Activation of the complete classical complement pathway can be determined by investigating binding of the Fc multimer to C1q, the first step in activation of the classical complement pathway, and formation of C4a, C5a or soluble or membrane bound C5b-9 complex, the final effector in the classical complement pathway. For example, an Fc multimer does not induce complete activation of the classical complement pathway if the protein binds C1q but soluble C5b-9 is essentially not formed, i.e. only less than 50% of the respective positive control is formed, preferably less than 40%, preferably less than 30%, preferably less than 20%, preferably less than 10%, more preferably less than 5%. Determining binding of an Fc multimer to C1q and induction of C5b-9 formation can be assessed as described below and in Example 7. Activation of the classical complement pathway can also be determined by assessing the generation of C4a, cleavage of C2, or formation of C3 convertase. For example, an Fc multimer does not induce activation of the complete classical complement pathway if it induces the generation of C4a but either does not induce cleavage of C2 or does not induce formation of C3 convertase. “Not induce” means less than 50%, preferably less than 40%, preferably less than 30%, preferably less than 20%, preferably less than 10%, more preferably less than 5% of the respective positive control is formed. Determining generation of C4a, cleavage of C2, and formation of C3 convertase can be assessed as described below and in Example 7.

The ability of an Fc multimer to bind C1q can be determined by an in vitro binding assay, such as an enzyme-linked immunosorbent assay (ELISA). For example, wells of a 96-well plate can be pre-coated with human C1q followed by the addition of Fc multimers. Purified peroxidase-labeled anti-human IgG conjugate can be added and bound conjugate can be visualized by using a color-producing peroxidase substrate, such as 3,3′,5,5′ tetramethylbenzidine (TMB). For example, the ability of an Fc multimer to bind C1q can be determined as shown in Example 7.

Activation of the classical complement pathway by an Fc multimer can be determined by in vitro assays and indicated by generation of C4a and soluble C5b-9. For example, different concentrations of an Fc multimer can be incubated in whole blood or serum for a predetermined period of time and any resulting generation of C4a or soluble C5b-9 (sC5b-9) can be determined by immunodetection, such as ELISA. Concentrations of Fc multimer used may be 0.01 mg/ml to 2 mg/ml, for example, 0.04 mg/ml, 0.2 mg/ml, or 1.0 mg/ml. For example, activation of the classical complement pathway can be determined as shown in Example 7.

Generation of C4a and sC5b-9 induced by an Fc multimer can be compared relative to the generation of these components induced by heat-aggregated gamma globulin (HAGG), a potent activator of the classical complement pathway. For example, this assay can be performed in whole blood. According to some embodiments, the Fc multimer induces less than 50% sC5b-9 generation, less than 40% sC5b-9 generation, less than 30% sC5b-9 generation, less than 20% sC5b-9 generation, or less than 10% sC5b-9 generation as compared to sC5b-9 generation induced by HAGG. In one embodiment, the Fc multimer induces less than 20% sC5b-9 generation in whole blood as compared to sC5b-9 generation induced by HAGG in whole blood. In another embodiment, the Fc multimer induces less than 10% sC5b-9 generation in whole blood as compared to sC5b-9 generation induced by HAGG in whole blood. In yet another embodiment, the Fc multimer induces no sC5b-9 generation. For example, induction of sC5b-9 generation can be determined as shown in Example 7.

The term “normal human serum activated with heat aggregated IgG” as used herein refers to a normal human serum sample where cleavage of nearly all C4 has been induced with heat aggregated IgG.

Activation of the classical complement pathway by an Fc multimer can also be determined by detecting C2 protein. If C2 protein is cleaved to C2a and C2b, the level of C2 protein decreases, indicating activation of the classical complement pathway. Different concentrations of an Fc multimer can be incubated in whole blood or serum for a predetermined period of time, for example 2 h, following which C2 protein levels can be determined by immunodetection, such as immunoblotting. Activation of the classical complement pathway is indicated by cleavage of the C2 protein. The level of C2 protein in normal human serum can be compared to the level of C2 protein resulting after pre-incubation with an Fc multimer to determine the amount of C2 cleavage, and therefore activation of the classical complement pathway. A known activator of the classical complement pathway, such as HAGG, can be used as a positive control for inducing cleavage of the majority of the C2 protein in normal human serum. The term “majority,” as used herein, is defined as comprising greater than 50%, greater than 60%, greater than 70%, greater than 80%, or greater than 90%. In some embodiments, the Fc multimer does not induce the cleavage of the majority of C2 protein. For example, cleavage of C2 protein can be determined as in Example 7.

Activation of the classical complement pathway by an Fc multimer can also be determined by assessing formation of C3 convertase. As described above, C3 convertase consists of the C2a and C4b subunits (C4b2a). If C2 protein is not cleaved to C2a and C2b, C3 convertase cannot be formed. As such, C3 convertase formation can be assessed as described above for determining C2 protein cleavage. In some embodiments, the Fc multimer does not induce formation of C3 convertase.

### Inhibition of the Classical Complement Pathway

Inhibition of the classical complement pathway by an Fc multimer can be determined by determining inhibition of C5a and sC5b-9 generation or by determining inhibition of cleavage of C2 protein. Different concentrations of the Fc multimer can be incubated in whole blood or serum with a known activator of the classical complement pathway. The level of sC5b-9 generated in the presence of an Fc multimer and a known activator of the classical complement pathway can then be compared to the level of sC5b-9 generated with the known activator of the classical complement pathway alone. The level of sC5b-9 generated can be determined as described above. The concentrations of Fc multimer used may be 0.01 mg/ml to 2 mg/ml, for example, 0.04 mg/ml, 0.2 mg/ml, or 1.0 mg/ml. The known activator of the classical complement pathway may be HAGG. The lower the level of sC5b-9 generated in the presence of an Fc multimer and an activator of the classical complement pathway is in comparison to the level of sC5b-9 generated in the presence of an activator of the classical complement pathway alone, the greater is the inhibition of sC5b-9 generation by the Fc multimer. For example, inhibition of sC5b-9 generation can be determined as in Example 7. In some embodiments, the Fc multimer inhibits greater than 50% sC5b-9 generation, greater than 60% sC5b-9 generation, greater than 70% sC5b-9 generation, greater than 80% sC5b-9 generation or greater than 90% sC5b-9 generation as compared to sC5b-9 generation induced by HAGG. In one embodiment, the Fc multimer inhibits greater than 80% of sC5b-9 generation induced by HAGG.

The term “inhibit,” as used herein, is defined as to suppress, restrict, prevent, interfere with, stop, or block.

Inhibition of cleavage of C2 protein can be similarly determined. Different concentrations of the Fc multimer can be incubated in whole blood or serum with a known activator of the classical complement pathway. The greater the level of C2 protein in the presence of an Fc multimer and a known activator of the classical complement pathway compared to the level of C2 protein in the presence of the known activator of the classical complement pathway alone, the greater is the inhibition of C2 cleavage by the Fc multimer. The level of C2 protein can be determined as described above. The concentrations of Fc multimer used may be 0.01 mg/ml to 2 mg/ml, for example, 0.04 mg/ml, 0.2 mg/ml, or 1.0 mg/ml. The known activator of the classical complement pathway can be HAGG. For example, inhibition of C2 cleavage can be determined as in Example 7. In some embodiments, the Fc multimer inhibits the cleavage of the majority of C2 protein by HAGG.

Inhibition of the classical complement pathway can also be determined using a hemolysis assay for the classical complement pathway using antibody-sensitized, or opsonized, erythrocytes. For example, sheep erythrocytes, or red blood cells, can be opsonized with rabbit anti-sheep antibodies. Normal human serum (NHS) will induce lysis of opsonized erythrocytes. Fc proteins can be pre-incubated with NHS and then added to the erythrocytes and incubated for 1 h at 37° C. The concentration of Fc construct can be from 1-1000 μg/ml, for example 2.5, 25, 50, 125, 250, or 500 μg/ml. Alternatively, Fc monomer can also be pre-incubated with NHS at the same concentrations as indicated for the Fc construct. After incubation, the mixture can be centrifuged and the degree of lysis can be determined by measuring the absorbance of released hemoglobin at 412 nm of the supernatant. For example, lysis of erythrocytes in a hemolysis assay for the classical complement pathway can be determined as described in Example 7.

Inhibition of the classical complement pathway by the Fc multimer can be indicated by reduced lysis of erythrocytes in the mixtures that contain Fc multimer compared to the mixtures that have NHS but not Fc multimer. Inhibition of lysis of opsonized red blood cells by an Fc multimer can also be compared to lysis of opsonized red blood cells in the presence of the Fc monomer. In some embodiments, the Fc multimer inhibits lysis of opsonized sheep red blood cells as compared to Fc monomer. In one embodiment, the Fc multimer inhibits lysis of opsonized sheep red blood cells by over 70% as compared to Fc monomer.

Other Effects Demonstrated for the Fc Multimers that Indicate their Therapeutic Benefit:

The therapeutic benefit of the Fc multimer in the treatment of auto-antibody-mediated autoimmune diseases is also illustrated by the effect on the clearance of a tracer antibody in mice expressing human FcRn. This shows that the Fc multimer is capable of blocking FcRn in vivo, and indicates that it will be potent in reducing auto-antibody levels in autoimmune disease.

The therapeutic benefit of the Fc multimer in the treatment of certain autoimmune diseases is also demonstrated by the effect on phagocytosis of IgG-coated beads by a human monocyte cell line (THP-1). This indicates that the Fc multimer is functionally blocking Fcγ receptors in vitro. This indicates that the Fc multimers will be capable to inhibit chronic inflammation, which should be beneficial in the treatment of certain autoimmune diseases.

The safety and therapeutic benefit of Fc multimers is demonstrated by the lack of activation of neutrophils and inhibition of activation demonstrated by Fc multimer.

The safety of the Fc multimers is also indicated by the lack of effect on liver function.

### EXAMPLES

### Example 1: Preparation of IgG1 Fc Multimers

Fc-μTP (FIG. 1A, left diagram) was generated by fusing the 18 amino acid residues (PTLYNVSLVMSDTAGTCY) of human IgM tail piece to the C-terminus of the constant region of human IgG1 Fc fragment (amino acid residues 216-447, EU numbering; UniProtKB—P01857). Fc-μTP-L309C (FIG. 1A, right diagram) was generated by mutating the Leu residue at 309 (EU numbering) of Fc-μTP to Cys. The DNA fragments encoding Fc-μTP and Fc-μTP-L309C were synthesized and codon-optimized for human cell expression by ThermoFisher Scientific (MA, USA). The DNA fragments were cloned into ApaLI and XbaI sites of pRhG4 mammalian cell expression vector using InTag positive selection method (Chen, C G et al, (2014). Nucleic Acids Res 42(4):e26; Jostock T, et al (2004). J. Immunol. Methods. 289:65-80). Briefly, Fc-μTP and Fc-μTP-L309C fragments were isolated by ApaLI and AscI digestion. A CmR InTag adaptor comprising of BGH polyA addition sites (BGHpA) and chloramphenicol resistance gene (CmR) was also isolated by AscI and SpeI digestion (Chen, C G et al, (2014). Nucleic Acids Res 42(4):e26). The Fc molecules and the CmR InTag adaptor were co-cloned into ApaLI and XbaI sites of pRhG4 vector using T4 DNA ligase. Positive clones were selected on agar plates containing 34 μg/ml chloramphenicol. Miniprep plasmid DNA was purified using the QIAprep Spin Miniprep kit (QIAGEN, Hilden, Germany) and sequence confirmed by DNA sequencing analysis. The restriction enzymes and T4 DNA ligases were purchased from New England BioLabs (MA, USA).

The transient transfection using Expi293™ Expression System (Life Technologies, NY, USA) was performed according to the manufacturer's instruction. Briefly, plasmid DNA (0.8 μg) was diluted in 0.4 ml Opti-MEM and mixed gently. Expifectamine 293 Reagent (21.6 μL) was diluted in 0.4 ml Opti-MEM, mixed gently and incubated for 5 min at room temperature. The diluted Expifectamine was then added to the diluted DNA, mixed gently and incubated at room temperature for 20-30 min to allow the DNA-Expifectamine complexes to form. The DNA-Expifectamine complex was then added to the 50 ml Bioreactor tube containing 6.8 ml of Expi293 cells (2×10⁷ cells). The cells were incubated in a 37° C. incubator with 8% CO₂ shaking at 250 rpm for approximately 16-18 h. A master mix consisting of 40 μl Enhancer 1 (Life Technologies, NY, USA), 400 μl Enhancer 2 (Life Technologies, NY, USA) and 200 μl of LucraTone™ Lupin was prepared and added to each Bioreactor tube. The cells were incubated for further 4 days in a 37° C. incubator with 8% CO₂ shaking at 250 rpm. Protein was harvested from supernatant centrifugation at 4000 rpm for 20 min and filtered into a clean tube using a 0.22 μm filter before HPLC quantitation and purification.

In order to produce IgG1 Fc multimers, the N-terminus of recombinant human IgG1 Fc was fused to the 18 amino acid tailpiece of IgM. The IgM tailpiece (pTP) promotes formation of pentamers and hexamers. The Fc fusion proteins were produced with either wild-type (VVT) human IgG1 Fc peptide (Fc-μTP) or a variant thereof with a point mutation of leucine to cysteine at residue 309 (Fc-μTP-L309C). The leucine 309 to cysteine point mutation (Fc-μTP-L309C) was expected to provide a more stable structure than the WT (Fc-μTP) due to the formation of covalent bonds between Fc molecules.

The Fc-μTP and Fc-μTP-L309C fusion monomeric subunits result from two peptides comprising the following regions:
Signal peptide residues 1-19
Hinge region of human IgG1 residues 20-34
Fc region of human IgG1 residues 35-251
Tailpiece of human IgM residues 252-269

The amino acid sequences for the Fc-μTP and Fc-μTP-L309C peptides are provided as SEQ ID NO:1 and SEQ ID NO:2, respectively. The nucleic acid coding sequences are provided as SEQ ID NO:9 and SEQ ID NO:10, respectively.

During expression, the signal peptide is cleaved off to form the mature Fc-μTP and Fc-μTP-L309C fusion peptides.

SDS-PAGE of the multimeric Fc proteins showed a laddering pattern for each preparation, corresponding to monomer, dimer, trimer, tetramer, pentamer and hexamers of the Fc construct. Fc-μTP-L309C, but not Fc-μTP, had a predominant band at the expected hexamer position, which was consistent with a more stable structure under the disruptive electrophoresis buffer conditions (FIG. 1B). Diagrams of the expected structures for the Fc-μTP and Fc-μTP-L309C hexamers are shown in FIG. 1A. Higher order structures, most likely dimers of hexamer, were also evident for Fc-μTP-L309C.

Multimerization of Fc-μTP-L309C and Fc-μTP was also examined with size exclusion chromatography (SEC) (FIG. 10) and asymmetric flow field-flow fractionation (A4F) (FIG. 1D) of the Fc multimer preparations, followed by U.V. absorbance measurement at 280 nm (A280,thin chromatogram) and multi-angle light scattering (MALS, bold line). Similar distribution patterns with a predominant hexamer peak (approximately 85% material) were observed for each of the Fc multimer preparations with each procedure (Table 1). This is in contrast to the distinct profiles by SDS-PAGE (FIG. 1B) suggesting the presence of non-covalent hexamers in the Fc-μTP preparation. The remaining material was mostly lower order (monomer, dimer, trimer) for Fc-μTP and higher order (dimers of hexamer) for Fc-μTP-L3090.

TABLE 1

| Construct | Technique | % monomer | % dimer | % trimer | % hexamer | % Multimer |
|---|---|---|---|---|---|---|
| Fc-μTP | SEC-MALS | 13 (73 kD) | | 2 (168 kD) | 84 (355 kD) | |
| | A4F-MALS | 10 (60 kD) | | | 87 (305 kD) | 3 (491 kD) |
| Fc-μTP-L309C | SEC-MALS | | 4 (114 kD) | 4 (211 kD) | 84 (383 kD) | 8 (745 kD) |
| | A4F-MALS | 2 (62 kD) | | | 83 (327 kD) | 15 (592 kD) |

Recombinant human IgG1 Fc monomer (SEQ ID NO:3) was also produced and used as a control.

The Fc proteins (Fc, Fc-μTP and Fc-μTP-L309C) were considered to be endotoxin-free based on their inability to stimulate NF-κB activation in THP1 cells (FIG. 2). The human monocytic cell line, THP1, was cultured in Roswell Park Memorial Institute (RPMI) 1640 medium containing 10% fetal calf serum (FCS), 1% (100 U/ml) penicillin/streptomycin. Cell culture medium was replaced approximately every 3 days. THP1XBlue cells were derived by stable transfection of THP1 cells with a reporter plasmid expressing a secreted embryonic alkaline phosphatase (SEAP) gene under the control of a promoter inducible by the transcription factor NF-κB. Upon stimulation, THP1XBlue cells activate NF-κB and subsequently the secretion of SEAP which is readily detectable using QUANTI-blue, as medium turns purple/blue in its presence. THP1XBlue cells express all TLRs, as determined by PCR, but respond only to TLR2, TLR2/1, TLR2/6, TLR4, TLR5 and TLR8. THP1XBlue cells are resistant to the selection marker Zeocin. Cells were cultured in RPMI 1640 medium containing 10% FCS, 0.5% (100 U/ml) penicillin/streptomycin, 100 μg/ml Normocin (Invivogen, San Diego, Calif.) and 200 μg/ml Zeocin (Invivogen). Cell culture medium was replaced approximately every 3 days. Lipopolysaccharide (LPS) was used as a positive control for NF-κB activation.

### Example 2: Binding of Fc Multimers to FcγRs and Primary Human Myeloid Cells

Fc-mediated effects are initiated through the binding of Fc to specific receptors on the surface of leukocytes. Both Fc-μTP and Fc-μTP-L309C hexamers bound the Fcγ receptors (FcγRs) CD16a (FcγRIIIa), CD32a (FcγRIIa), CD32b/c (FcγRIIb/c) and CD64 (FcγRI) as determined by Biacore. Additionally, both Fc-μTP and Fc-μTP-L309C hexamers displayed fast on-rates and slow off-rates compared to recombinant Fc monomer, consistent with an avidity effect through their binding to multiple immobilized FcγR molecules (FIG. 3A). No major differences in binding response were observed between the two recombinant Fc molecules.

The binding of the Fc hexamers to human myeloid cell lines and primary cells was evaluated by flow cytometry and immunofluorescence (IF), using fluorescently-labelled anti-IgG Abs recognizing the Fc portion for detection. Cells were incubated with the Fc proteins for 2 h at 4° C., washed four times with PBS containing 0.1% BSA (Sigma) and 0.01% NaN₃ (Sigma) and stained with a FITC-labelled mAb against human IgG or respective isotype control Ab (BD) for 45 min at 4° C. Stained cells were washed a further four times and then analyzed by flow cytometry with a BD FACSCanto II flow cytometer (BD Biosciences AG, Allschwil, Switzerland) and the data evaluated using FlowJo.

The human monocytic cell line THP1 expresses the FcγRs CD64 and CD32 but not CD16, on its surface (FIG. 4) and so it was used to evaluate Fc construct binding in a whole cell system. Both Fc-μTP and Fc-μTP-L309C bound to a similar degree to THP1 cells, with each showing a greater binding signal than IVIG (FIG. 3B). In view of the detection method, the differences in maximum response may reflect the greater number of Fc epitopes present on the multimeric Fc molecules compared to IVIG. Fc-μTP and Fc-μTP-L309C also bound to primary human neutrophils to a markedly greater extent than IVIG or recombinant Fc monomer, with the VVT construct showing the highest level of binding (FIG. 3C).

For primary human macrophages, peripheral blood monocytes were first differentiated in vitro into M1 (inflammatory) and M2 (anti-inflammatory) lineage macrophages, then incubated with the Fc proteins and examined by IF staining for IgG1 Fc. Fc-μTP and Fc-μTP-L309C each co-stained strongly with the two primary human macrophage lineage cells, while Fc monomer stained weakly (particularly for M1 macrophages) and IVIG was intermediate (FIG. 3D).

Binding of the Fc proteins to FcγR was quantified in a whole cell system of CD16-transfected NFAT-bla Jurkat cells in the presence of AF488-labelled IgG1. Jurkat NFAT-bla CD16 cells in assay medium (PBS+2% FCS) were plated (1×10⁵/well) in U-bottom wells of a 96-well plate containing mixtures of AF488-labelled human IgG1 (3.75 pg/ml; 25 nM) and increasing concentrations of Fc-containing test molecules. The assay mixture was incubated for 2 h at room temperature with constant shaking, then cells were quickly washed in ice-cold medium, before fixation for 15 min on ice (BD Cytofix). Cells were resuspended in medium, and the cell-bound fluorescence in each well was read on a Fortessa flow cytometer. Ki values, which indicate the concentration (in nano-molar [nM]) of test sample required for displacement of 50% of the labelled IgG1 from the transfected cells, were determined. Both Fc-μTP and Fc-μTP-L309C displaced IgG1 from binding to the Jurkat cells to a similar degree (Ki of 1.47 nM and 1.43 nM, respectively) and the Ki of each was >100 fold lower than that of IgG1 or its recombinant Fc component (Ki of 225 nM and 240 nM respectively) (FIG. 3E).

### Example 3: Binding of Fc Multimers to the Neonatal FcR

The neonatal FcR (FcRn) in epithelial cells binds pinocytosed IgG at acidic pH and redirects it away from the lysosomal degradative pathway to be released extracellularly, thereby extending the serum half-life of IgG. To determine whether the Fc multimers, which bear multiple IgG1 Fc moieties, can also bind to FcRn, they were first examined by Octet analysis using immobilized human FcRn at pH 6.0. The Octet assays were performed as described previously (Neuber, T et al (2014) MAbs 6(4) 928-942) and measured in a 96-well format on an Octet QKe device (FortéBio Inc., Menlo Park, Calif., USA). The assays were analyzed and fitted with the Octet Software 7.0.1.1 (ForteBio Inc.). Both Fc-μTP and Fc-μTP-L309C bound to FcRn and showed slower off-rates compared to recombinant IgG1 Fc (FIG. 5A), suggesting an avidity effect.

To confirm that binding could occur in a whole cell system, binding of the Fc proteins to a human FcRn-transfected 293FS cell line (293FS+FcRn+β2m (clone #2)) was assessed in serum-free assay medium (PBS at pH 5.5). Cells were plated (2×10⁵/well) and co-incubated with AF488-labelled IgG1 (0.5 μg/ml; 3.33 nM) and increasing concentrations of Fc proteins. The assay mixture was incubated for 1-2 h at room temperature with constant shaking. After the incubation, the cell-bound fluorescence in each well was read on a Fortessa flow cytometer and Ki values were determined. Interestingly, a differential response was obtained between the VVT and mutant Fc multimer, with Fc-μTP displacing IgG1 more readily from the FcRn than Fc-μTP-L309C (FIG. 5B). Fc-μTP-L309C showed a similar mean Ki to IgG1 (approximately 200 nM).

Functional impact on the half-life of endogenous immunoglobulin was demonstrated by enhanced clearance of a tracer monoclonal antibody (mAb) in vivo. IVIG [2 g/kg], Fc-μTP-L309C [200 mg/kg] or PBS were administered i.p. into human FcRn transgenic mice. Five minutes later, 5 mg/kg of a human monoclonal IgG1, specific for human IL-3 Receptor (CSL360), was injected i.v. Levels of tracer mAb in serum were measured at indicated time points (FIG. 17) by MSD-based immunoassay at a constant serum dilution. Clearance of the tracer mAb was accelerated by IVIG and Fc-μTP-L309C, which indicates saturation of FcRn by these molecules.

### Example 4: Pharmacokinetics of Fc Multimers

To examine whether the differential binding of Fc-μTP and Fc-μTP-L309C to FcRn would be reflected in differences in serum half-life, the pharmacokinetics of the Fc proteins were compared to that of IVIG following a single i.v. injection into human FcRn-transgenic mice and VVT rats. In mice, all doses were 100 mg/kg. In rats, IVIG was administered at a dose of 250 mg/kg and Fc-μTP and Fc-μTP-L309C were administered at a dose of 25 mg/kg. In both mice and rats, both Fc proteins displayed a more rapid clearance from the serum than IVIG, indicative of a shorter serum half-life (FIG. 6A and FIG. 6B, respectively). There was no difference between the VVT and mutant constructs.

The differences between the two administration routes, i.e. i. v. versus s.c. has been evaluated. The bioavailability of s.c. is approx. 25% compared to i. v.

### Example 5: Fc Multimers Provide Therapeutic Effects in Acute Inflammatory Ab-Induced Arthritis

### A. Fc Multimers Induce Reduction of Clinical Scores

The Fc proteins were examined for therapeutic effect in the collagen Ab-induced arthritis (CAbIA) model in mice. CAbIA is a model that is dependent on both FcR engagement and the complement system (Banda, N K et al, (2007) J Immunol 179(6), 4101-9; Kagari T et al (2003), J Immunol 170(8) 4318-4324). Disease was induced in WT Balb/c mice as shown in FIG. 7A. Mouse monoclonal anti-type II collagen 5 clone mAb cocktail kit was purchased from Chondrex Inc. (Redmond, Wash.). On day 0 mice were injected i.p. with 2 mg mAb cocktail followed by 50 μg LPS i.p. on day 3. Mice were monitored for up to 14 days for clinical signs of arthritis. A clinical score was assigned as follows: 0—normal; 0.5—swelling confined to digits; 1—mild paw swelling; 2—marked paw swelling; 3—severe paw swelling and/or ankylosis. For therapeutic treatments, mice that showed signs of arthritis at day 6 (i.e. clinical score of 1 or more) were randomized and given a single intraperitoneal (i.p.) injection of Fc-μTP, Fc-μTP-L309C, IVIG, or PBS. Fc-μTP and Fc-μTP-L309C were administered at a dosage of 200 mg/kg, IVIG at 2 g/kg. For each mouse, the clinical score represents the cumulative clinical score for each paw each day starting on day 6 and up to day 14. Mean clinical scores were also calculated by averaging the clinical score from each of day 7 through day 14. Mice administered PBS served as an untreated control. The therapeutic effect of Fc-μTP and Fc-μTP-L309C administered s.c. was evaluated (FIG. 15). Fc-μTP and Fc-μTP-L309C given s.c. demonstrated a similar therapeutic effect as when administered i.p.

Both Fc-μTP and Fc-μTP-L309C rapidly reduced the clinical signs of disease, with effects being evident within 24 h (day 7) and sustained through day 14 (FIG. 7B). By 24 h after treatment (day 7) with either Fc-μTP or Fc-μTP-L309C, clinical scores were reduced by 50% compared to mice administered PBS (untreated). Clinical scores of mice administered either Fc-μTP or Fc-μTP-L309C remained over 50% lower than untreated mice through day 14. The resulting mean clinical scores over days 7 to 14 were over 50% lower in Fc-μTP- and Fc-μTP-L309C-treated mice than untreated mice. Conversely, the mice administered IVIG did not show a significant reduction in clinical score compared to untreated mice until day 14 and the mean clinical score from days 7-14 of IVIG-treated mice was not significantly different from that of untreated mice.

### B. Fc Proteins Induce Reduction of Joint Infiltrating Cells

Joints of arthritic mice were processed for further assessment of markers of inflammation. Patellas and surrounding soft tissue (excluding fat) were removed from both rear limbs and placed in RPMI+5% FCS on ice for 60 min. The medium was then removed, centrifuged and the supernatant (joint wash) stored at −30° C. to await subsequent analysis. The washed patellas and cell pellets were combined for each individual mouse and digested for 30 min at 37° C. in a shaking incubator (140 cycles/min) with 1 mg/ml collagenase (CLS-1, 250 U/mg; Worthington Biochemical Corp, Lakewood, N.J.) and 0.1 mg/ml DNAse 1 (type IV from bovine pancreas, 2100 kU/mg; Sigma). The digests were strained (70 μm cutoff), washed and resuspended in PBS+2% FCS for cell counts, cytospins and flow cytometry.

Single cell suspensions of peripheral blood and joint digests were resuspended in PBS containing 2% (v/v) FCS. Cells were stained with the following mAbs: FITC-conjugated anti-mouse Ly6G (1A8; BioLegend), eFluor450-conjugated anti-mouse CD11b (M1/70; eBioscience), APC.Cy7-conjugated anti-mouse CD45 (30-F11, BD Pharmingen), PE-conjugated anti-mouse CXCR2 (242216, R&D Systems), APC-conjugated anti-mouse CXCR4 (2B11, eBioscience), PE.Cy7-conjugated anti-mouse CD62L (MEL-14, eBioscience). 7AAD (BD Pharmingen) was used to exclude dead cells and live cells were analyzed on a BD Fortessa using FlowJo software. Mice treated with Fc-μTP- and Fc-μTP-L309C had over 50% fewer infiltrating (CD45+) cells recovered from knee joints at day 8 of disease compared to untreated (PBS) control mice (FIG. 7C).

### C. Fc Proteins Induce Reduction of Histological Scores

Histopathology of the arthritic joints was also assessed. At days 8 and 14, mice were killed and the left rear paws were fixed in 10% neutral buffered formalin, decalcified and embedded in paraffin. Sagittal tissue sections were stained with H&E and scored blinded to the treatment groups. The ankle joints were globally scored for three features (exudate—presence of inflammatory cells within the joint space; synovitis—the degree of synovial membrane thickening and inflammatory cell infiltration; tissue destruction—cartilage and bone erosion and invasion), each out of five (0—normal, 1—minimum, 2—mild, 3—moderate, 4—marked, 5—severe), and these were summed for a total score out of fifteen.

Histopathology of the arthritic joints confirmed the clinical assessment and joint cell evaluations with reduced inflammatory cell infiltrate, synovitis and cartilage and bone destruction evident in the Fc-μTP- and Fc-μTP-L309C-treated mice as early as day 8 (FIG. 7D and FIG. 7E). At day 8, the Fc proteins induced a greater than 25% reduction of histological scores compared to mice administered PBS. By day 14, histological scores of mice treated with Fc-μTP- and Fc-μTP-L309C were greater than 50% lower than in untreated mice administered PBS (FIG. 7E). Conversely, the histological scores of mice administered IVIG were not significantly different than those of untreated mice.

### D. Fc Proteins Reduce Levels of Cytokines and Chemokines in Arthritic Joints

The local inflammatory response in the arthritic joints was further evaluated by assessing cytokine and chemokine levels. Day 8 joint washes, described above, were evaluated by protein array using a commercial kit and analysis was performed according the manufacturers protocol (Kit MCYTOMAG-70K from Millipore). Both Fc proteins reduced the levels of the inflammatory cytokines IL-6, LIF and G-CSF but not IL-9 (FIG. 8). The chemokines IP-10 (CXCL10), MCP-1 (CCL2), KC (CXCL1), and MIP-2 (CXCL2) were each significantly reduced, while MIG (CXCL9), eotaxin (CCL11) and RANTES (CCLS) were not. In contrast, IVIG had minimal impact on the cytokine/chemokine levels at day 8, with only levels of MIP-2 being significantly reduced. This could reflect the slower kinetics of the IVIG response, which was previously reported in this model (Campbell I K et al., Journal of Immunology. 2014; 192(11): 5031-5038).

### Example 6: Impact of Fc Multimers on the Complement Cascade in Arthritic Mouse Joints

To determine whether the Fc proteins could be alleviating disease through an effect on the complement system, levels of components of the complement pathway were determined in day 8 joint washes from the arthritic mice described above. Levels of C1q, C3, and C5a were determined by ELISA. All three complement components tested were elevated in the joint washes of arthritic mice compared to naive mice (FIG. 9). Fc-μTP and Fc-μTP-L309C each reduced the levels of C1q, C3 and C5a detectable in the joint washes to a similar degree. IVIG treatment resulted in a marked elevation in C1q and slightly reduced C3, while C5a levels were unaffected.

### Example 7: Fc Multimers Inhibit the Classical Complement Pathway in Human Serum and Whole Blood

### A. Effect of Fc Proteins on Complement System

The effect of the Fc proteins on the complement system was investigated using human in vitro models. The effect of Fc proteins on the function of the classical (CP), lectin (LP), and alternative pathways (AP) was examined in the Wieslab® Complement System Screen (Euro-Diagnostica, Malmo, Sweden), which is an enzyme immunoassay for the specific detection of the three pathways with deposition of C5b-9 as a common read-out. Further dilutions were made according to the instructions, i.e. 1:100 for the classical and lectin pathway and 1:18 for the alternative pathway. Results were compared relative to activation in normal human serum (NHS).

Both Fc-μTP and Fc-μTP-L309C inhibited the complete activation of the classical complement pathway but demonstrated no significant effect on the lectin or alternative pathways (FIG. 11A). Conversely, Fc monomer had no effect on any of the complement pathways. The preincubation was conducted using 10% normal human serum with the addition of 90% PBS containing the test sample (e.g. Fc-μTP-L309C or control samples), and incubation for 5-15 minutes at 37 degrees. Then the sample was diluted 1:10 with assay diluent buffer provided by the Wieslab® Complement Kit (Lectin Pathway). Subsequently the assay was performed according to the manufacturer's instructions.

However, it was surprising that no effect on the lectin pathway was observed. Therefore, we proceeded to optimize the conditions described above as follows. Optimal C4 cleavage was achieved using 20% normal human serum, 10% PBS containing the test sample (e.g. Fc-μTP-L309C or control sample) and 70% Gelatin Veronal Buffer (GVB²⁺·0.15 mM CaCl₂, 0.5 mM MgCl₂) for one hour at 37 degrees. Then the sample was diluted 1:20 with assay diluent buffer provided by the Wieslab® Complement Kit (Lectin Pathway). Subsequently the assay was performed according to the manufacturer's instructions. As shown in FIGS. 20A and B, optimized preincubation resulted in much higher levels of cleaved C4 (as shown by generation of C4a) than the previous above described non-optimized conditions (i.e. 10% NHS and 90% PBS) but even incubated for one hour (instead of 5-15 minutes). Subsequently, a potent inhibitory effect on the lectin pathway was observed due to C4 depletion. However, inhibition of the classical pathway was already observed using “non-optimized”buffer conditions as there is a very strong binding to C1q (described in Example 7C) which is essential for activation of the classical pathway.

As shown in FIG. 21A, Fc-μTP-L309C demonstrated a potent inhibitory effect on activation of the classical and lectin pathway, whereas no inhibition of the alternative pathway was observed. In the same samples tested in the Wieslab® Kits, C4a and C5a were measured. Optimized preincubation conditions induced optimal cleavage of C4, whereas no generation of C5a was observed (FIG. 21B). Specificity of Wieslab® was demonstrated using sera depleted for the indicated complement protein (FIG. 21C). As shown in FIG. 21C, depletion of C4 results in inhibition (or non-functionality) of the classical and lectin pathways, whereas the alternative pathway is still active.

### B. Fc Proteins Inhibit Hemolysis by Classical Complement Pathway

To investigate Fc proteins effects on the classical pathway, sheep erythrocytes (Siemens) were sensitized with rabbit anti-sheep antibodies (Ambozeptor 6000; Siemens) and diluted to 4×10⁸ cells/mL GVB²⁺ (GVB, 0.15 mM CaCl₂, 0.5 mM MgCl₂). To assess inhibition of hemolysis by Fc monomer, Fc-μTP and Fc-μTP-L309C, the recombinant proteins were pre-incubated in 1/40 diluted NHS (30 min at RT) and subsequently added to the erythrocytes at a 1/1 (v/v) ratio and incubated during 1 h at 37° C. in a microtiter-plate shaking device. The concentrations of Fc monomer, Fc-μTP and Fc-μTP-L309C used were 2.5, 25, 50, 125, 250, and 500 μg/ml. After adding ice-cold GVBE (GVB, 10 mM EDTA) and centrifugation (5 min at 1250×g, 4° C.), hemolysis was determined in the supernatant by measuring the absorbance of released hemoglobin at 412 nm and compared to hemolysis induced by NHS.

To investigate Fc construct effects on the alternative pathway, rabbit erythrocytes (Jackson Laboratories) were washed and diluted to 2×10⁸ cells/mL GVB/MgEGTA (GVB, 5 mM MgEGTA). To assess inhibition of hemolysis by Fc monomer, Fc-μTP and Fc-μTP-L309C, the recombinant proteins were pre-incubated in 1/6 diluted NHS (30 min at RT) and subsequently added to the erythrocytes at a 2/1 (v/v) ratio and incubated during 1 h at 37° C. in a microtiter-plate shaking device. The concentrations of Fc monomer, Fc-μTP and Fc-μTP-L309C used were 250, 500, and 1000 μg/ml. After adding ice-cold GVBE and centrifugation (10 min at 1250×g), hemolysis was determined in the supernatant by measuring the absorbance of released hemoglobin at 412 nm and compared to hemolysis induced by NHS. Fc monomer had no effect on hemolysis of sheep red blood cells via the classical complement pathway and levels of lysis were equivalent to levels with NHS alone. Conversely, Fc-μTP and Fc-μTP-L309C both greatly inhibited lysis induced by NHS (FIG. 10A). At concentration equal to or above 2.5 μg/ml, both Fc-μTP and Fc-μTP-L309C inhibited lysis by over 70% compared to the amount of lysis that occurred in the presence of the same concentration of Fc monomer (as shown in Table 2). Neither the Fc monomer nor either of the Fc proteins inhibited the alternative complement pathway in rabbit red blood cells (FIG. 10B).

TABLE 2
TABLE 2. CH50

| | %-lysis of RBC | | |
|---|---|---|---|
| concentration [μg/mL] | Fc monomer | Fc-μTP | Fc-μTP-L309C |
| 0 (Serum; pos ctr) | 100 | 100 | 100 |
| 500 | 96.0 ± 7.2 | 14.4 ± 1.7 | 13.8 ± 1.8 |
| 250 | 101.6 ± 4.7 | 14.3 ± 1.5 | 13.5 ± 1.5 |
| 125 | 102.1 ± 7.0 | 15.3 ± 1.4 | 14.6 ± 1.5 |
| 50 | 100.9 ± 5.6 | 15.6 ± 1.8 | 15.2 ± 1.7 |
| 25 | 98.8 ± 8.7 | 17.4 ± 3.2 | 16.5 ± 2.9 |
| 2.5 | 102.8 ± 7.6 | 21.9 ± 4.2 | 17.7 ± 2.7 |
| 1.25 | 106.7 ± 3.0 | 75.8 ± 4.4 | 33.9 ± 18.0 |
| 0.625 | 106.2 ± 12.1 | 95.1 ± 2.7 | 80.4 ± 2.5 |
| 0.3125 | 98.7 ± 8.3 | 94.2 ± 3.6 | 92.6 ± 3.2 |
| 0.15625 | 100.6 ± 8.0 | 96.8 ± 5.4 | 99.6 ± 4.2 |

Results are expressed as mean ± standard deviation (SD)

### C. Fc Multimer Binding to C1q

The effects of the Fc proteins on the classical complement pathway were examined by evaluating binding to C1q. Binding of Fc proteins to C1q was analyzed by ELISA (Inova Diagnostics, San Diego, Calif.). Wells were precoated with human C1q and Fc proteins (<16 μg/mL) were added to allow binding. After washing of the wells to remove all unbound protein, purified peroxidase-labelled goat anti-human IgG conjugate was added. Unbound protein was removed by a further wash step and bound conjugate was visualized with 3,3′,5,5′ tetramethylbenzidine (TMB) substrate. The Fc multimers demonstrated a high degree of binding to C1q compared to the Fc monomer (FIG. 11B).

### D. Fc Multimer Effect on the Classical Complement Pathway

The effects of Fc construct on complement were assessed in human whole blood and human serum. For the assay whole of NHS was diluted 1:5 with GVB²⁺ Buffer (Complement Tech). Activation of human complement in serum was analyzed by the generation of C3a, C4a and C5a by ELISA (Quidel, San Diego, Calif.). Analysis of complement activation in human whole blood was based on anticoagulation with recombinant hirudin, which is a highly specific thrombin inhibitor that does not influence complement activation. Like for NHS, whole blood was diluted 1:5 in GVB²⁺ Buffer. Complement activation was analyzed by the generation of C4a and sC5b-9 by ELISA (Quidel).

Incubation for 2 hours of human whole blood with different concentrations of Fc-μTP or Fc-μTP-L309C induced cleavage of the complement protein C4 into C4a, but no increase sC5b-9 was observed (FIG. 11C). Similar results were found in human serum (FIG. 12A). Neither Fc monomer nor IVIG induced cleavage of C4 or the formation of sC5b-9 (FIG. 11C).

### E. Fc Multimers Inhibit Activation of the Complete Classical Complement Pathway

Heat aggregated IgG (HAGG) is a known activator of the classical complement pathway. Pre-incubation of human serum or whole blood with Fc-μTP or Fc-μTP-L309C did not affect generation of C4a by HAGG (not shown) but fully prevented further down-stream activation of the classical complement pathway, as shown by reduced levels of sC5b-9 (FIG. 11D) and C5a (FIG. 12B). Fc multimers inhibited HAGG-induced sC5b-9 generation in human whole blood by over 80%. Conversely, Fc monomer and IVIG had no effect on induction of sC5b-9 levels by HAGG (FIG. 11D). These results indicate that hexameric Fc induced activation of the classical pathway by binding to C1q, with activation of the C1 complex and cleavage of C4 but apparently no formation of the C3 convertase (C4b2a), as no downstream complement activation could be detected.

To form the C3 convertase, C4 and C2 need to be cleaved by C1 s. As shown in FIG. 11C, C4 was cleaved by incubation of human whole blood with the Fc proteins. To investigate C2 cleavage, levels of C2 were measured in human serum incubated for 2 h with Fc monomer, Fc-μTP, or Fc-μTP-L309C by Western blot. Equal amounts of human serum (1 μl serum per lane) were loaded and separated by 4-12% SDS-PAGE (Invitrogen). Equal loading was verified by staining the extracted gel with GelCode Blue Stain Reagent (ThermoScientific). Gels were then transferred to PVDF membranes (Invitrogen). Afterwards, the membranes were blocked (ON at 4° C.) with Superblock (ThermoScientific), and incubated (2 h at RT) with the primary mouse antibodies against complement protein C2 (R&D Systems, diluted 1:1′000 in Superblock), Membranes were washed (PBS+0.05% Tween20) and incubated (2 h at RT) with horseradish peroxidase-coupled goat polyclonal anti-mouse IgG (Dako, diluted 1:1,000 in Superblock). Finally, the membranes were developed with a chemiluminescence detection kit (SuperSignal West Pico chemiluminescent, ThermoScientific).

Following incubation of human serum with HAGG, the classical pathway was activated and C2 was cleaved as shown by the disappearance of the C2 band at approximately 100 kD (molecular weight of uncleaved C2) (FIG. 11E). Neither of the Fc multimers nor Fc monomer induced noticeable cleavage of C2. When serum was incubated with HAGG and either Fc-μTP or Fc-μTP-L309C, C2 cleavage was inhibited (FIG. 11E). Conversely, addition of Fc monomer had no effect on cleavage of C2 induced by HAGG. These data suggest that, in the fluid phase, the Fc multimers inhibited C2 cleavage, thereby preventing formation of the C3 convertase and subsequent activation steps of the classical complement pathway.

### Example 8: Fc Multimers Inhibit Complement Deposition on Endothelial Cells

To investigate whether the Fc multimers prevented complement deposition on cells, an assay was used to test for deposition of complement fragments on human umbilical vein endothelial cells (HUVEC). HUVEC were purchased from Lonza (Lonza, Visp, Switzerland) and cultured according to the manufacturer's description. For analysis of C3b deposition, HUVEC were opsonized with an anti-CD105 (Endoglin) mAb (Abcam) prior to incubation with normal human serum (Quidel) diluted in veronal buffer saline (1:5) for 30-60 min at 37° C. Cells were washed and C3b deposition was detected using a FITC-conjugated anti-C3c mAb (Dako) and quantified by flow cytometry.

Incubation of opsonized HUVEC with serum containing Fc-μTP or Fc-μTP-L309C resulted in a dose-dependent inhibition of C3b deposition whereas monomeric Fc did not prevent deposition of C3b (FIG. 11F). Overall, these data suggest that inhibition of full activation of the classical complement pathway may be one of the effector mechanisms by which Fc-μTP and Fc-μTP-L309C reduce inflammation.

### Example 9: Fc Multimers Inhibit FcγR Expression In Vivo

The effect of Fc proteins on FcγR expression in the CAbIA arthritis model described above was investigated. As described above, mice with CAbIA were treated at day 6 with PBS (untreated control), Fc-μTP, or Fc-μTP-L309C (FIG. 7A). Neutrophils and monocyte/macrophages were obtained from the joints and blood at day 8. Flow cytometry showed a significant reduction in CD16/32 (FcγRIII/FcγRII) surface expression by neutrophils and monocyte/macrophages from both joints and blood taken from mice administered Fc-μTP or Fc-μTP-L309C compared to untreated (PBS) arthritic mice (FIG. 13A). CD16/32 (FcγRIII/FcγRII) expression on joint neutrophils, joint monocyte/macrophages, and blood neutrophils was over 50% lower in arthritic mice that were treated with Fc-μTP or Fc-μTP-L309C compared to arthritic mice administered PBS. CD16/32 (FcγRIII/FcγRII) expression on blood monocytes was over 25% lower in mice that were treated with Fc-μTP or Fc-μTP-L309C compared to arthritic mice administered PBS.

The high affinity FcγR, CD64 (FcγRI), was also significantly reduced on joint neutrophils (greater than 50%) and blood monocyte/macrophages (greater than 75%) in mice treated with Fc-μTP or Fc-μTP-L309C compared to untreated arthritic mice (FIG. 13A). These data could suggest either down-modulation of FcγR expression, or FcR blockage by the Fc-μTP or Fc-μTP-L309C.

### Example 10: Fc Multimers Inhibit FcγR Function In Vitro

Human in vitro systems were used to examine the effects of the Fc proteins on FcγR function. Human neutrophils were isolated from the buffy coat fraction of anticoagulated blood samples obtained from healthy volunteers (Regional Red Cross Blood Donation Center, Bern, Switzerland) by dextran sedimentation (MW 450-650 kD) followed by Ficoll gradient centrifugation. Neutrophils were obtained from the sediment after hypotonic lysis of remaining erythrocytes. Respiratory burst was measured by pipetting test articles in triplicates into wells of a microtiterplate followed by the purified neutrophils previously added to a luminol solution (Sigma). Chemiluminescence was recorded at 37° C. during 90 min, the area under the signal-to-time curve was calculated and results expressed as RLU (relative light units). As a positive control rabbit erythrocytes (Charles River) previously incubated with human IgG (CSL Behring) were used. Anti-rabbit erythrocyte specific human IgG antibodies specifically bind to the erythrocytes, which therefore activate human neutrophils in a FcγR specific manner.

To measure inhibition of respiratory burst of neutrophils activated by human IgG treated rabbit erythrocytes, neutrophils were preincubated with inhibitors (Fc monomer, Fc-μTP or Fc-μTP-L309C) for 15 minutes at 37° C., subsequently the luminol solution and the IgG treated rabbit erythrocytes were added and chemiluminescence was recorded as described above.

Human neutrophils were incubated either with Fc-μTP or Fc-μTP-L309C in the presence and absence of IgG-coated rabbit RBCs and the respiratory burst was used as a measure of neutrophil activation. The Fc proteins failed to stimulate respiratory burst under conditions where IgG-coated rabbit RBCs could (FIG. 13B). Moreover, both Fc multimers inhibited the activation of the respiratory burst in response to IgG-coated rabbit RBCs (FIG. 13C).

Second, the effect of the Fc proteins on FcγR function was evaluated using Ab-dependent cell-mediated cytotoxicity (ADCC) of opsonized (anti-D-treated O+) human erythrocytes as a readout. Peripheral blood mononuclear cells (PBMC) were isolated from buffy coats obtained from healthy volunteers with blood group O (Regional Red Cross Blood Donation Center, Bern, Switzerland) by Ficoll gradient centrifugation. Subsequently, PBMC were depleted of monocytes by adherence on polystyrene. The resulting lymphocytes were considered effector cells. As target cells, purified human Rh(D)+ red blood cells (RBC) of blood group O volunteers (Regional Red Cross Blood Donation Center, Bern, Switzerland) were used. The RBCs were intracellularly labelled with CFSE (Molecular Probes) and then opsonized with anti-D (Rhophylac CSL Behring AG).

To assess inhibition of ADCC, lymphocytes were preincubated with inhibitors (IVIG, Fc-μTP or Fc-μTP-L309C) for 30 min at 37° C., then aliquots of the mixtures were added in triplicates to wells of target cell loaded microtiterplates at an effector:target ratio of 1:1. Microtiterplates were incubated overnight at 37° C. and 5% CO₂, then centrifuged and the supernatant discarded. The RBC were washed once with 0.9% NaCl before lysis of the sediment with 1% TritonX100. Aliquots of the lysates were pipetted into the wells of a microtiterplate and fluorescence was determined (excitation 480 nm/emission 535 nm). ADCC was calculated using control samples (labelled erythrocytes without incubation with anti-D) and 100% lysis samples (erythrocytes treated with TritonX100).

Fc-μTP and Fc-μTP-L309C potently inhibited ADCC, whereas IVIG had minimal effect (FIG. 13D). Taken together, the data suggest that blockade of FcγRs is another possible anti-inflammatory effector mechanism of Fc-μTP or Fc-μTP-L309C.

Fc-μTP or Fc-μTP-L309C, administered as a bolus at 200 mg/kg, prevented up-regulation of C5aR (CD88) on peripheral monocytes in the CAbIA arthritis model at day 8 as shown by flowcytometry (FIG. 13E).

The functional impact on FcγR mediated phagocytosis has been evaluated. THP1 cells were incubated with IgG coated FITC-labeled latex beads for 3 hours in presence of IVIG or Fc-μTP-L309C. Afterwards uptake of beads was analyzed by flow cytometry. As shown in FIG. 17, Fc-μTP-L309C was much more potent than IVIG in blocking phagocytosis of IgG coated latex beads.

### Example 11: Effect of Fc Proteins In Vitro on Purified Leukocytes and Human Whole Blood

The effect of the Fc proteins on cytokine secretion by purified human leucocytes (buffy coats) was assessed after 6 h culture in the presence and absence of HAGG using Luminex array according the manufacturer instructions (Invitrogen). Cytokine/chemokine levels in culture supernatants or plasma (for human whole blood assay) were measured using a commercial human cytokine magnetic 30-plex panel (Invitrogen Life Technologies, Paisley, UK) according to the manufacturer's instructions. The panel consisted of the following analytes: GM-CSF, IL-2, IL-1β, TNF-α, IL-4, IL-6, MIP-1α, MIP-1β, Eotaxin, RANTES, MIG, VEGF, HGF, EGF, IL-8, IL-17, IL-1RA, IL-12 (p40/p70) IL-13, FGF-Basic, IFN-γ, G-CSF, MCP-1, IL-7, IL-15, IFN-α, IL-2R, IP-10, IL-10, IL-5. Out of the 30 cytokines/chemokines, 25 were not induced by HAGG. Subsequently, the 5 cytokines/chemokines (IL-1RA, MCP-1, MIP-1(3, RANTES, and IL-8) which were induced by HAGG were analyzed. The Fc proteins induced secretion of IL-1RA, MCP-1, MIP-1β, RANTES, and IL-8 but to a considerable lower amount than HAGG.

The effect on cytokine and chemokine secretion of Fc monomer, Fc-μTP and Fc-μTP-L309C was analyzed using human whole blood. Hirudin, a specific thrombin inhibitor, which does not interfere with the complement system, was used as an anticoagulant. Human whole blood was incubated with the Fc-μTP and Fc-μTP-L309C overnight. The following analytes were measured by luminex according the manufactures instructions (Invitrogen): GM-CSF, IL-2, IL-1β, TNF-α, IL-4, IL-6, MIP-1a, MIP-1β, Eotaxin, RANTES, MIG, VEGF, HGF, EGF, IL-8, IL-17, IL-1RA, IL-12 (p40/p70) IL-13, FGF-Basic, IFN-γ, G-CSF, MCP-1, IL-7, IL-15, IFN-α, IL-2R, IP-10, IL-10 and IL-5. Fc-μTP or Fc-μTP-L309C induced detectable levels of IL-1β, TNF-α, IL-6, MIP-1α, MIP-1β, Eotaxin, RANTES, MIG, HGF, IL-8, IL-1RA, IL-12 (p40/p70), IL-13, IFN-γ, G-CSF, MCP-1,IL-15, IFN-α, IL-2R, IP-10 and IL-10. The following analytes were induced to a considerable lower amount by Fc-μTP and Fc-μTP-L309C (used highest concentration of 1 mg/mL) than by LPS (100 ng/mL; Sigma, E. coli 0111:B4, γ-irradiated; Cat. No. L4391): IL-1β, TNF-α, MIP-1a, MIP-1β, IL-1RA, IL-12 (p40/p70), G-CSF and IL-10.

The response was unlikely to be due to contaminating platelets, which express FcγRIIA (CD32A) in humans, since none of the Fc proteins activated platelets, measured by the up-regulation of P-selectin (CD62P) using flow cytometry (FIG. 17). Platelet activation was analyzed by measurement of the P-selectin (CD62P) expression on platelet surface by flow cytometry (BD FACSCanto II). In Brief, PRP was obtained from citrate stabilized human blood by centrifugation at 300×g for 10 min. at 22° C. Aggrastat at 2.5 μg/ml (final concentration) was added to PRP to avoid platelet aggregation. The PRP was incubated with Fc monomer, Fc-μTP or Fc-μTP-L309C for 15 minutes. As positive controls, ADP (16 μM) and/or Convulxin (20 ng/ml) were used.

The effect on Ca²⁺ flux or mobilization of calcium has been evaluated in a FACS assay using purified human leukocytes. The cells were loaded with a calcium indicator dye which allows detecting intracellular calcium mobilization by fluorescence. Briefly, cells were incubated with the calcium indicator dye Cal-520 for 60 min at 37° C., washed and re-suspended in 5 ml HBSA/Hepes. Samples were equilibrated before Fc-μTP and Fc-μTP-L309C or heat aggregated IgG (control) were added and fluorescence recorded at room temperature for the indicated times. Cellular subsets were distinguished using forward (FSC) and side scatter (SSC). As shown in FIG. 18, Fc-μTP and Fc-μTP-L309C did not induce calcium mobilization in neutrophils.

### Example 12: Effect of Fc Proteins on Cytokines and Liver Toxicity In Vivo

The in vivo effects of the Fc proteins on cytokines and liver toxicity were investigated in wild type rats (CD strain). Rats were administered a 25 mg/kg dose of Fc-μTP or Fc-μTP-L309C, a 250 mg/kg dose of IVIG, or 0.9% NaCl. Cytokine/chemokine levels in rat plasma were measured using a commercial rat cytokine magnetic 24-plex panel (BioRad) according to the manufacturer's instructions. The panel consisted of the following analytes: EPO, G-CSF, GM-CSF, GRO/KC, IFN-γ, IL-1α, L-1β, IL-2, IL-4, IL-5, IL-6, IL-7, IL-10, IL-12p70, IL-13, IL-17A, IL-18, M-CSF, MCP-1, MIP-1α, MIP-3α, RANTES, TNF-α, VEGF. Liver toxicity markers were measured using a commercial rat 5-plex panel (Merck Millipore) according to the manufacturer's instructions. The panel included of the following analytes: Liver-Type Arginase 1 (ARG1), Aspartate transaminase 1 (GOT1), a-glutathione S-transferase (GSTa), Sorbitol Dehydrogenase (SDH), 5′-Nucleotidase/CD73 (5′-NT) and the analysis was performed according the manufacturer's instructions (Merck Millipore). As shown in Table 3, none of the measured 24 cytokines was significantly up-regulated by the Fc multimers at the used dose of 25 mg/kg given intravenously. Moreover, only Aspartate transaminase 1 (GOT1) and 5′-Nucleotidase/CD73 (5′-NT) was detected but a very low levels which were comparable to IVIG (as shown in Table 4).

Furthermore, cytokines and markers of liver toxicity were evaluated in FcRn^{-/-}, hFcRN^{tg/+} mice receiving 100 mg/kg i.v. of IVIG, Fc-μTP or Fc-μTP-L309C. Cytokines/chemokine levels in mouse plasma were measured using a commercial mouse cytokine luminex kit (Merck Millipore) according to the manufacturer's instructions. The panel consisted of the following analytes: G-CSF, GM-CSF, IFNγ, IL-1α, IL-1β, IL-2, IL-6, IL-9, IL12p70, LIX/CXCL5, IL-15, IL-17, IP-10/CXCL10, KC/CXCL1, M-CSF, MIP-2/CXCL2/3, MIG/CXCL9, Rantes/CCL5, VEGF. To evaluate liver toxicity the following markers were evaluated using COBAS: ALT, AST, LDH, bilirubin, C-reactive protein. For all markers measured the detected levels were comparable those observed after administration of IVIG.

TABLE 3
IN-VIVO CYTOKINES IN RATS

| | | conc. in [pg/mL] | | | |
|---|---|---|---|---|---|
| Analyte | Timepoint (in h) | NaCl 0.9% | IVIG | Fc-μTP | Fc-μTP-L309C |
| IL1a | all | n.d. | n.d. | n.d. | n.d. |
| IL1b | 0 | 0.35 | n.a. | n.a. | n.a. |
| | 0.083 | n.a. | n.d. | 0.05 | n.d. |
| | 0.5 | n.a. | n.d. | 0.07 | 0.04 |
| | 2 | n.a. | 0.06 | 0.07 ± 0.01 | 0.05 |
| | 6 | n.a. | 0.07 ± 0.01 | 0.14 ± 0.11 | 0.08 ± 0.01 |
| | 24 | n.a. | 0.12 ± 0.05 | 0.2 ± 0.18 | n.d. |
| | 72 | n.a. | n.d. | n.d. | n.d. |
| | 168 | n.a. | n.d. | n.d. | n.d. |
| IL2 | all | n.d. | n.d. | n.d. | n.d. |
| IL4 | all | n.d. | n.d. | n.d. | n.d. |
| IL5 | 0 | 0.07 | n.a. | n.a. | n.a. |
| | 0.083 | n.a. | 0.15 ± 0.03 | 0.16 ± 0.10 | 0.05 ± 0.04 |
| | 0.5 | n.a. | 0.14 ± 0.01 | 0.17 ± 0.13 | 0.11 ± 0.01 |
| | 2 | n.a. | 0.16 | 0.19 ± 0.04 | 0.14 ± 0.01 |
| | 6 | n.a. | 0.2 ± 0.01 | 0.18 ± 0.02 | 0.19 ± 0.04 |
| | 24 | n.a. | 0.18 ± 0.06 | 0.13 ± 0.01 | 0.07 ± 0.03 |
| | 72 | n.a. | 0.13 | 0.12 ± 0.02 | 0.1 ± 0.01 |
| | 168 | n.a. | 0.23 | 0.13 ± 0.06 | 0.13 ± 0.05 |
| IL6 | all | n.d. | n.d. | n.d. | n.d. |
| IL7 | 0 | 0.23 ± 0.23 | n.a. | n.a. | n.a. |
| | 0.083 | n.a. | 0.04 | 0.15 ± 0.14 | 0.06 |
| | 0.5 | n.a. | 0.05 | 0.05 ± 0.04 | 0.13 ± 0.11 |
| | 2 | n.a. | 0.05 ± 0.3 | 0.17 | 0.04 |
| | 6 | n.a. | 0.05 | 0.02 | 0.21 ± 0.15 |
| | 24 | n.a. | 0.03 | 0.11 | 0.03 |
| | 72 | n.a. | 0.06 ± 0.05 | 0.03 ± 0.01 | 0.97 ± 1.04 |
| | 168 | n.a. | 0.09 | 0.11 ± 0.08 | 0.05 ± 0.05 |
| IL10 | 0 | n.d. | n.a. | n.a. | n.a. |
| | 0.083 | n.a. | n.d. | 0.17 | n.d. |
| | 0.5 | n.a. | 0.69 ± 0.42 | 1.34 ± 0.1 | 0.88 ± 0.04 |
| | 2 | n.a. | 0.09 ± 0.01 | 0.11 ± 0.01 | 0.08 |
| | 6 | n.a. | n.d. | n.d. | n.d. |
| | 24 | n.a. | 0.1 | 0.07 | n.d. |
| | 72 | n.a. | n.d. | 0.11 | n.d. |
| | 168 | n.a. | 0.12 ± 0.05 | 0.1 | 0.09 |
| IL12p70 | all | n.d. | n.d. | n.d. | n.d. |
| IL13 | all | n.d. | n.d. | n.d. | n.d. |
| IL17A | all | n.d. | n.d. | n.d. | n.d. |
| IL18 | 0 | n.d. | n.a. | n.a. | n.a. |
| | 0.083 | n.a. | 0.6 ± 0.31 | 0.45 | 0.54 ± 0.24 |
| | 0.5 | n.a. | n.d. | 0.64 | n.d. |
| | 2 | n.a. | 0.86 | n.d. | 0.61 ± 0.27 |
| | 6 | n.a. | n.d. | 0.36 | n.d. |
| | 24 | n.a. | 0.82 | n.d. | 0.57 ± 0.17 |
| | 72 | n.a. | n.d. | 0.43 ± 0.01 | 0.46 ± 0.08 |
| | 168 | n.a. | 0.48 ± 0.06 | n.d. | 0.92 |
| TNFa | all | n.d. | n.d. | n.d. | n.d. |
| IFNg | all | n.d. | n.d. | n.d. | n.d. |
| MCP1 | 0 | 0.91 ± 0.1 | n.a. | n.a. | n.a. |
| | 0.083 | n.a. | 1.07 ± 0.06 | 1.37 ± 0.31 | 0.69 ± 0.26 |
| | 0.5 | n.a. | 1.46 ± 0.10 | 1.07 ± 0.42 | 1.13 ± 0.25 |
| | 2 | n.a. | 2.60 ± 0.95 | 4.53 ± 1.34 | 2.77 ± 0.21 |
| | 6 | n.a. | 3.65 ± 0.29 | 3.44 ± 0.91 | 4.14 ± 1.07 |
| | 24 | n.a. | 1.32 ± 0.26 | 1.90 ± 0.13 | 1.01 ± 0.55 |
| | 72 | n.a. | 0.68 ± 0.08 | 0.61 ± 0.23 | 0.71 ± 0.15 |
| | 168 | n.a. | 1.09 ± 0.02 | 1.05 ± 0.13 | 0.70 ± 0.21 |
| MIP2 | 0 | n.d. | n.a. | n.a. | n.a. |
| | 0.083 | n.a. | n.d. | n.d | n.d. |
| | 0.5 | n.a. | 0.10 | 0.09 | 0.12 |
| | 2 | n.a. | 0.04 | 0.03 | n.d. |
| | 6 | n.a. | 0.04 | n.d. | n.d. |
| | 24 | n.a. | 0.03 | 0.04 | n.d. |
| | 72 | n.a. | n.d. | n.d. | n.d. |
| | 168 | n.a. | n.d. | n.d. | n.d. |
| MIP3α | 0 | n.d. | n.a. | n.a. | n.a. |
| | 0.083 | n.a. | 0.03 ± 0.01 | 0.07 | n.d. |
| | 0.5 | n.a. | 0.04 | 0.06 ± 0.06 | 0.03 |
| | 2 | n.a. | 0.04 | n.d. | 0.03 ± 0.01 |
| | 6 | n.a. | 0.02 | n.d. | 0.02 |
| | 24 | n.a. | 0.05 | 0.03 | n.d. |
| | 72 | n.a. | 0.03 ± 0.01 | 0.04 ± 0.01 | 0.03 ± 0.01 |
| | 168 | n.a. | 0.06 ± 0.01 | 0.04 | 0.04 |
| RANTES | 0 | n.d. | n.a. | n.a. | n.a. |
| | 0.083 | n.a. | 0.14 ± 0.07 | 0.42 | n.d. |
| | 0.5 | n.a. | 0.17 | 0.57 | 0.24 ± 0.09 |
| | 2 | n.a. | 0.13 | n.d. | 0.10 |
| | 6 | n.a. | n.d. | n.d. | 0.14 |
| | 24 | n.a. | 0.25 | n.d. | n.d. |
| | 72 | n.a. | 0.12 | 0.14 | 0.12 ± 0.04 |
| | 168 | n.a. | 0.32 ± 0.02 | 0.25 | 0.18 ± 0.06 |
| G-CSF | all | n.d. | n.d. | n.d. | n.d. |
| GM-CSF | all | n.d. | n.d. | n.d. | n.d. |
| M-CSF | 0 | 0.60 | n.a. | n.a. | n.a. |
| | 0.083 | n.a. | 0.15 ± 0.04 | 0.19 ± 0.02 | 0.11 ± 0.03 |
| | 0.5 | n.a. | 0.14 ± 0.01 | 0.17 ± 0.01 | 0.16 ± 0.02 |
| | 2 | n.a. | 0.20 ± 0.10 | 0.19 ± 0.03 | 0.13 ± 0.03 |
| | 6 | n.a. | 0.18 ± 0.01 | 0.23 ± 0.01 | 0.21 ± 0.04 |
| | 24 | n.a. | 0.16 ± 0.06 | 0.20 ± 0.06 | 0.12 ± 0.02 |
| | 72 | n.a. | 0.13 ± 0.06 | 0.15 ± 0.03 | 0.14 ± 0.02 |
| | 168 | n.a. | 0.16 ± 0.05 | 0.15 ± 0.05 | 0.10 ± 0.03 |
| VEGF | all | n.d. | n.d. | n.d. | n.d. |
| EPO | 0 | n.d. | n.a. | n.a. | n.a. |
| | 0.083 | n.a. | 0.28 | 0.34 | 0.24 ± 0.19 |
| | 0.5 | n.a. | 0.14 | 0.42 | 0.11 |
| | 2 | n.a. | 0.21 ± 0.09 | n.d. | 0.36 |
| | 6 | n.a. | n.d. | n.d. | n.d. |
| | 24 | n.a. | 0.31 | n.d. | 0.36 |
| | 72 | n.a. | n.d. | 0.14 ± 0.03 | 0.16 ± 0.01 |
| | 168 | n.a. | 0.25 ± 0.11 | 0.21 | 0.49 |
| GRO/KC | 0 | 0.08 ± 0.08 | n.a. | n.a. | n.a. |
| | 0.083 | n.a. | 0.03 ± 0.01 | 0.07 ± 0.07 | 0.03 ± 0.02 |
| | 0.5 | n.a. | 0.05 ± 0.01 | 0.04 ± 0.01 | 0.09 ± 0.06 |
| | 2 | n.a. | 0.13 ± 0.03 | 0.17 ± 0.04 | 0.12 ± 0.04 |
| | 6 | n.a. | 0.05 ± 0.01 | 0.02 ± 0.01 | 0.10 ± 0.08 |
| | 24 | n.a. | 0.03 ± 0.01 | 0.05 ± 0.04 | 0.02 ± 0.01 |
| | 72 | n.a. | 0.06 ± 0.02 | 0.03 ± 0.01 | 0.33 ± 0.28 |
| | 168 | n.a. | 0.05 ± 0.02 | 0.06 ± 0.05 | 0.04 ± 0.03 |

Results are expressed as mean ± standard deviation (SD)
n.d.: not detectable
n.a.: not applicable

TABLE 4
IN-VIVO LIVER TOXICITY MARKERS IN RATS

| | | conc. in [pg/mL] | | | |
|---|---|---|---|---|---|
| Analyte | Timepoint (in h) | NaCl 0.9% | IVIG | Fc-μTP | Fc-μTP-L309C |
| Liver-type | 0 | n.d. | n.a. | n.a. | n.a. |
| arginase 1 | 0.083 | n.d. | 29.80 | n.d. | n.d. |
| (ARG1) | 0.5 | n.d. | n.d. | n.d. | n.d. |
| | 2 | n.d. | n.d. | n.d. | n.d. |
| | 6 | n.d. | n.d. | n.d. | n.d. |
| | 24 | n.d. | n.d. | n.d. | n.d. |
| | 72 | n.d. | n.d. | n.d. | n.d. |
| | 168 | n.d. | n.d. | n.d. | n.d. |
| Aspartate | 0 | 13.72 | n.a. | n.a. | n.a. |
| transaminase | 0.083 | n.a. | 49.54 ± 50.28 | 15.69 ± 4.63 | 12.37 ± 1.08 |
| (GOT1) | 0.5 | n.a. | 9.93 ± 0.38 | 13.18 ± 1.43 | 12.55 ± 0.82 |
| | 2 | n.a. | 14.80 ± 2.28 | 20.98 ± 6.52 | 14.17 ± 0.62 |
| | 6 | n.a. | 14.39 ± 1.71 | 17.98 ± 0.20 | 14.16 ± 1.05 |
| | 24 | n.a. | 14.66 ± 9.95 | 24.46 ± 15.94 | 12.37 ± 1.78 |
| | 72 | n.a. | 19.37 ± 3.42 | 12.46 ± 0.68 | 12.41 ± 2.07 |
| | 168 | n.a. | 13.92 ± 2.38 | 15.60 ± 1.40 | 15.07 ± 1.89 |
| α-glutathianone | 0 | n.d. | n.a. | n.a. | n.a. |
| S-transferase | 0.083 | n.d. | 16.24 | n.d. | n.d. |
| (GSTa) | 0.5 | n.d. | n.d. | n.d. | n.d. |
| | 2 | n.d. | n.d. | n.d. | n.d. |
| | 6 | n.d. | n.d. | n.d. | n.d. |
| | 24 | n.d. | n.d. | n.d. | n.d. |
| | 72 | n.d. | n.d. | n.d. | n.d. |
| | 168 | n.d. | n.d. | n.d. | n.d. |
| sorbitol | 0 | n.d. | n.a. | n.a. | n.a. |
| dehydrogenase | 0.083 | n.d. | 451.68* | n.d. | n.d. |
| (SDH) | 0.5 | n.d. | n.d. | n.d. | n.d. |
| | 2 | n.d. | n.d. | n.d. | n.d. |
| | 6 | n.d. | n.d. | n.d. | n.d. |
| | 24 | n.d. | n.d. | n.d. | n.d. |
| | 72 | n.d. | n.d. | n.d. | n.d. |
| | 168 | n.d. | n.d. | n.d. | n.d. |
| 5′-Nucleotidase | 0 | 1.50 | n.a. | n.a. | n.a. |
| (5′-NT/CD73) | 0.083 | n.a. | 28.69 ± 37.99 | 2.11 ± 0.50 | 1.58 ± 0.65 |
| | 0.5 | n.a. | 2.44 | 1.41 ± 0.28 | 1.58 ± 0.35 |
| | 2 | n.a. | 3.57 | 1.82 ± 0.77 | 1.74 ± 0.38 |
| | 6 | n.a. | 1.82 | 1.74 ± 0.14 | 1.33 |
| | 24 | n.a. | 1.52 ± 0.60 | 1.82 ± 0.25 | 1.65 ± 0.14 |
| | 72 | n.a. | 2.08 ± 1.40 | 2.07 ± 0.70 | 1.70 ± 0.18 |
| | 168 | n.a. | 2.07 ± 0.35 | 2.75 ± 2.06 | 2.15 ± 0.57 |

*only one value detectiable of the duplicate
n.a.: not applicable
n.d.: not detectable
Results are expressed as mean ± standard deviation (SD)

### Example 13: Fc Multimers Provide Therapeutic Effects in a Mouse Model of Myasthenia Gravis

Protective efficacy of Fc-μTP and Fc-μTP-L309C is tested in a mouse model of myasthenia gravis. Experimental autoimmune myasthenia gravis (EAMG) in mice is induced by immunizing mice with acetyl-cholin receptor (ACHR), for example purified from the electric organs of Torpedo californica by affinity chromatography, emulsified in Complete Freund's Adjuvans s.c. on day 1 followed by a boost with AChR in Incomplete Freund's Adujvans on day 26 after first immunization. Fc-μTP and Fc-μTP-L309C are applied prophylactically or therapeutically. The clinical signs of EAMG are scored, for example as described in M. Thiruppathi et al, J. Autoimmunity 2013: For clinical examination, mice are observed on a flat platform for a total of 2 min. They are then exercised by gently dragging them suspended by the base of the tail across a cage top grid repeatedly (20-30 times) as they attempted to grip the grid. They are then placed on a flat platform for 2 min and again observed for signs of EAMG. Clinical muscle weakness is graded as follows: grade 0—mouse with normal posture, muscle strength, and mobility at baseline and after exercise; grade 1—normal at rest but with muscle weakness characteristically shown by a hunchback posture, restricted mobility, and difficulty in raising the head after exercise; grade 2—grade 1 symptoms without exercise during observation period; grade 3—dehydrated and moribund with grade 2 weakness; and grade 4—dead.

Treatment with Fc-μTP and Fc-μTP-L309C significantly improves the clinical course of EAMG. Autoantibodies and inflammatory markers are down-modulated. Treatment is at least as effective as IVIg in suppressing EAMG.

### Example 14: Fc Multimers Provide Therapeutic Effects in a Mouse Model of Scleroderma

Protective efficacy of Fc-μTP and Fc-μTP-L309C is tested in a mouse model of scleroderma. Experimental scleroderma in mice is induced for example as described in Ruzek et al, Arth Rheumat 2004:50; 4; 1319-31 Spleens are harvested from B10.D2 and the tissue is dissociated into single cells by appropriate methods. Red blood cells are lysed and 2-5×107 B10.D2 (for induction of GVH) are injected intravenously into recipient BALB/c RAG-2 KO mice. Over the course of 5-9 weeks symptoms and histopathological modifications reminiscent of human scleroderma occur such as increase of dermal thickening, especially of the extremities, progressive fibrosis of internal organs, vasoconstriction and altered expression of vascular markers in skin and internal organs, inflammation in skin and internal organs, and autoantibody generation. Fc-μTP and Fc-μTP-L309C are applied prophylactically or therapeutically.

Treatment with Fc-μTP and Fc-μTP-L309C significantly mitigates/improves the histological modifications of the skin and internal organs and improves vascular and inflammatory modifications.

### Example 15: Fc Multimers Provide Therapeutic Effects in a Mouse Model of Auto-Immune Kidney Disease (Induced by Anti-Glomerular Base Membrane Autoantibodies)

Protective efficacy of Fc-μTP and Fc-μTP-L309C is tested in a mouse model of complement-mediated disease, for example kidney disease mediated by anti-glomerular base membrane autoantibodies. Anti-glomerular antibodies are purified from plasma of rabbits immunized with mouse glomeruli. Mice are injected i.v. with rabbit anti-glomerular antibodies on day 0, anti-rabbit IgG is injected on day 6 to crosslink the anti-glomerular antibodies. Acute and chronic albuminurias are assessed as a measure of kidney diseases on day 7 and 30, respectively. Fc-μTP and Fc-μTP-L309C are applied prophylactically or therapeutically.

Treatment with Fc-μTP and Fc-μTP-L309C significantly reduces albuminuria.

1. A hexameric protein, comprising six Fc fusion monomers, wherein each Fc fusion monomer comprises two polypeptide chains of SEQ ID NO:1, SEQ ID NO:2, or variants of SEQ ID NO:1 or SEQ ID NO:2 with up to 5 conservative amino acid changes, wherein the protein does not comprise an Fab polypeptide.

2. The hexameric protein of claim 1, wherein the protein binds complement component C1q, and wherein protein binding to C1q does not induce activation of the complete cascade of the classical complement pathway.

3. The hexameric protein of claim 2, wherein the protein binding to C1q, cleaves C4 but does not induce cleavage of the majority of C2.

4. The hexameric protein of claim 2 or 3, wherein the protein does not induce cleavage of C2.

5. The hexameric protein of claim 2, wherein the protein inhibits cleavage of the majority of C2 induced by heat aggregated IgG incubated with normal human serum.

6. The hexameric protein of claim 2, wherein the protein binding to C1q does not result in formation of C3 convertase.

7. The hexameric protein of claim 2, wherein 1 mg/ml of the protein incubated with whole blood induces less than 20% of soluble C5b-9 generation as compared to soluble C5b-9 generation induced by heat-aggregated IgG incubated with whole blood.

8. The hexameric protein of claim 2, wherein the protein incubated with whole blood induces less than 10% soluble C5b-9 generation as compared to soluble C5b-9 generation induced by heat-aggregated IgG incubated with whole blood.

9. The hexameric protein of claim 2, wherein the protein inhibits C5b-9 generation.

10. The hexameric protein of claim 2, wherein the protein inhibits C5b-9 generation induced by heat-aggregated IgG incubated with whole blood.

11. The hexameric protein of claim 2, wherein the protein activates the classical complement pathway by less than 20% as compared to normal human serum activated with heat aggregated IgG.

12. The hexameric protein of claim 1, wherein administration of 200 mg/kg of the protein at day 6 in a mouse anti-collagen antibody-induced arthritis model induces:
a) a reduction in the clinical score at any of days 7 to 14;
b) a reduction in the mean clinical score calculated from days 7 to 14;
c) a reduction in the number of CD45+ cells recovered from knee joints at day 8; or
d) a reduction of histological score of ankle joints at day 8 or day 14, wherein mice administered the hexameric protein are compared to untreated arthritic mice.

13. The hexameric protein of claim 1, wherein administration of 200 mg/kg of the protein at day 6 in a mouse anti-collagen antibody-induced arthritis model induces:
a) a greater than 50% reduction in the clinical score at any of days 7 to 14;
b) a greater than 50% reduction in the mean clinical score calculated from days 7 to 14;
c) a greater than 50% reduction in the number of CD45+ cells recovered from knee joints at day 8; or
d) a greater than 25% reduction of histological score of ankle joints at day 8 and/or a greater than 50% reduction of histological score of ankle joints at day 14, wherein mice administered the hexameric protein are compared to untreated arthritic mice.

14. The hexameric protein of claim 1, wherein the protein inhibits lysis of opsonized sheep red blood cells in a hemolysis assay for the classical complement pathway as compared to a recombinant Fc monomer comprising two polypeptides of SEQ ID NO:3.

15. The hexameric protein of claim 1, wherein at a concentration of 0.5 mg/ml, the protein inhibits lysis of opsonized sheep red blood cells in a hemolysis assay for the classical complement pathway by over 70% as compared to a recombinant Fc monomer comprising two polypeptides of SEQ ID NO:3.

16. The hexameric protein of claim 1, wherein the protein induces a reduction of FcγRII expression or FcγRIII expression on neutrophils or monocytes.

17. The hexameric protein of claim 1, wherein administration of 200 mg/kg of the protein at day 6 in a mouse anti-collagen antibody-induced arthritis model induces a greater than 50% reduction in FcγRII levels or FcγRIII levels on neutrophils or monocytes at day 8, wherein mice administered the hexameric protein are compared to untreated arthritic mice.

18. The hexameric protein of claim 1, wherein the protein inhibits upregulation of C5aR (CD88) on monocytes.

19. The hexameric protein of claim 1, wherein administration of 200 mg/kg of the protein at day 6 in a mouse anti-collagen antibody-induced arthritis model induces a reduction of CD64 levels on monocytes at day 8, wherein mice administered the hexameric protein are compared to untreated arthritic mice.

20. A method of treating an autoimmune or inflammatory disease in a subject in need thereof, comprising administering to the subject a therapeutically effective amount of a pharmaceutical composition comprising the hexameric protein of claim 1.

21. The method of claim 20, wherein the pharmaceutical composition is administered intravenously or non-intravenously.

22. The method of claim 21, wherein the pharmaceutical composition is administered subcutaneously.

23. The method of claim 20, wherein the autoimmune or inflammatory disease is chosen from immune cytopenia, Guillain-Barré syndrome, Kawasaki disease, chronic inflammatory demyelinating polyneuropathy, myasthenia gravis, inflammatory neuropathy, neuromyelitis optica, other autoimmune channelopathies, autoimmune epilepsy, dermatomyositis, polimyositis, pemphigus, pemphigoid, systemic lupus erythematosus, transplantation, reperfusion injury and rheumatoid arthritis.

24. The method of claim 20, wherein the pharmaceutical composition is administered at a dosage of 10 mg/kg to 1000 mg/kg.


SEQUENCE LISTING (10 sequences)

SEQ ID NO: 1 - protein, 250 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 2 - protein, 250 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 3 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 19 aa, Homo sapiens
MGWSCIILFLVATATGVHS

SEQ ID NO: 5 - protein, 15 aa, Homo sapiens
EPKSCDKTHTCPPCP

SEQ ID NO: 6 - protein, 18 aa, Homo sapiens
PTLYNVSLVMSDTAGTCY

SEQ ID NO: 7 - protein, 269 aa, Homo sapiens
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 8 - protein, 269 aa, Homo sapiens
MGWSCIILFLVATATGVHSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 9 - DNA, 750 bp, Homo sapiens
gagcccaagagctgcgacaagacccacacctgtcccccttgtcctgcccctgaactgctgggcggacctagcgtgttcctgttccccccaaagcccaaggacaccctgatgatctcccggacccccgaagtgacctgcgtggtggtggatgtgtcccacgaggaccctgaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtcactgagcctgagccccggcaagcccaccctgtacaatgtgtccctcgtgatgagcgacaccgccggcacctgttac

SEQ ID NO: 10 - DNA, 750 bp, Homo sapiens
gagcccaagagctgcgacaagacccacacctgtcccccttgtcctgcccctgaactgctgggcggacctagcgtgttcctgttccccccaaagcccaaggacaccctgatgatctcccggacccccgaagtgacctgcgtggtggtggatgtgtcccacgaggaccctgaagtgaagtttaattggtacgtggacggcgtggaagtgcataacgccaagaccaagcccagagaggaacagtacaacagcacctaccgggtggtgtccgtgctgaccgtgtgccaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtcactgagcctgagccccggcaagcccaccctgtacaatgtgtccctcgtgatgagcgacaccgccggcacctgttac